(19) United States
(12) Patent Application Publication
(10) Pub. No.: US 2016/0304887 A1
(43) Pub. Date: Oct. 20, 2016

(54) Introducing or Inactivating Female Fertility in Filamentous Fungal Cells

(71) Applicants: Technische Universitat Wein, Wien (AT); OREGON STATE UNIVERSITY, Corvallis, OR (US); ACADEMIA SINICA, Taipei (TW)

(72) Inventors: Monika Schmoll, Korneuburg (AT); Doris Tisch, Ternitz (AT); Andre Schuster, Neufeld an der Leitha (AT); Michael Freitag, Corvallis, OR (US); Kyle Pomraning, Richland, WA (US); Ting Fang Wang, Tapei City (TW)

(73) Assignee: Technische Universitat Wein, Wein (AT)

(21) Appl. No.: 15/103,554

(22) PCT Filed: Dec. 10, 2013
(86) PCT No.: PCT/EP2014/077273

§ 371 Date: Jun. 10, 2016

(30) Foreign Application Priority Data
Dec. 10, 2013 (EP) 13196402.5

(51) Int. Cl. C12N 15/80; C12P 21/00; C07K 14/37; C12N 1/14; C12R 1/885

(52) U.S. Cl. CPC C12N 15/80; C12N 1/14; C12R 1/885; C07K 14/37; C12P 21/00

(57) ABSTRACT

The present invention relates to a female fertile variant strain of filamentous fungus derived from a female sterile parental strain which comprises at least one of the six female fertility specific gene alleles or derivatives thereof comprising the functional characteristics of these alleles (FS_4-9).

### STATEMENT AS TO RIGHTS TO INVENTIONS MADE UNDER FEDERALLY-SPONSORED RESEARCH AND DEVELOPMENT

This invention was made with Government support under Contract DE-AC0576RLO1830 awarded by the U.S. Department of Energy. The Government has certain rights in the invention.

### REFERENCE TO A SEQUENCE LISTING

This application contains a Sequence Listing in computer readable form, which is incorporated herein by reference.

### DESCRIPTION

1. Field of the Invention

The present invention relates to a female fertile variant strain of a filamentous fungus derived from a female sterile parental strain which comprises at least one of the six female fertility specific gene alleles or derivatives thereof comprising the functional characteristics of these alleles (FS_4-9).

2. Background Art

Sexual development represents one of the most important accomplishments in evolution. Organisms from fungi to humans exchange and combine genomes with compatible partners for improved fitness and survival in the often harsh conditions of their natural habitat. Thereby, the alterations in the genomes brought about by the process of sexual development can be the only chance of survival under changing environmental conditions.

In fungi, sexual development occurs between compatible mating partners under specific conditions concerning nutrient availability, temperature, humidity, pH and light (Debuchy et al, 2010; Moore-Landecker, 1992). Fungi can be self-fertile (homothallic) meaning that one strain is capable of sexual reproduction even in solo culture. In contrast, self-sterile (heterothallic) fungi require two compatible partners for sexual development to occur. In pseudo homothallic fungi, nuclei of both mating types are present in one spore and enable mating (Ni et al, 2011).

Heterothallic fungi can have bipolar mating types, which are prevalent in ascomycetes, where two different sequences (called “idiomorphs”, comparable to sex chromosomes in higher organisms) occupy one and the same genomic region and thereby define the mating type of this fungus as MAT1-1 or MAT1-2 (Debuchy & Turgeon, 2006; Metzenberg & Glass, 1990). Within this locus different transcriptional activator genes are present, which are responsible for mating type dependent gene expression and hence for the phenotype reflecting the “gender” of a strain.

Fungi are hermaphrodites, which independently from their mating type can assume the female role (production of reproductive structures to be fertilized) or the male role (providing spores for fertilization of female reproductive structures). Consequently, for sexual development to occur, one of the mating partners has to act as a male and the other has to act as a female (Debuchy et al, 2010).

Trichoderma reesei is nowadays one of the most prolific fungal industrial workhorses for production of cell wall degrading enzymes and heterologous performance proteins (Schuster & Schmoll, 2010). After its isolation from degraded equipment of the US army during World War II, the potential of T. reesei to degrade cellulosic material was recognized, but only one strain, QM6a, made it to the research laboratories of industry and academia. Hence, all T. reesei strains used in research and industry today are derivatives of QM6a.

Despite several attempts, crossing of T. reesei under laboratory conditions was not achieved for decades and this fungus was even considered an asexual clonal line of the genus (Kuhls et al, 1996). Nevertheless, molecular methods confirmed Hypocrea jecorina as the sexual form (teleomorph) of T. reesei, although this relationship was suggested already very early in the history of research with this genus (Kuhls et al, 1996; Tulasne & Tulasne, 1865).

### SUMMARY OF INVENTION

It is an object of the present invention to restore female fertility in a female sterile parent filamentous fungus by introducing the functional alleles which correlate with female fertility.

Thus, the present invention relates to a female fertile variant strain of a filamentous fungus comprising at least one of the six female fertility specific alleles (FS4-9).

Therefore, the present invention relates to a female fertile variant strain of a filamentous fungus derived from a female sterile parental strain which comprises at least one, at least two, at least three, at least four, at least five or at least six of the gene alleles of SEQ ID NO:1, SEQ ID NO:3, SEQ ID NO:5, SEQ ID NO:7, SEQ ID NO:9 or SEQ ID NO:11 or a functional fragment or a derivative thereof.

A defect in female fertility in another fungal species, which is dependent on the same six genes or a subset thereof, could be repaired by introducing said functional alleles into said female sterile strain. Female fertility/sterility is known for many fungi for which a sexual cycle is known, including Trichoderma sp, Aspergillus sp. and Neurospora sp.

One aspect of the invention relates to a variant strain as described above, wherein the filamentous fungus is a filamentous heterothallic ascomycete, preferably selected from Trichoderma sp., Aspergillus sp. or Neurospora sp.

A further aspect of the invention refers to the variant strain as described above, wherein the filamentous fungus is Trichoderma reesei.

A further aspect of the invention refers to the variant strain as described above, wherein the Trichoderma reesei is QM6a.

A further aspect of the invention refers to the variant strain as described above, wherein the variant strain comprises at least one gene encoding at least one heterologous protein.

A further aspect of the invention refers to an isolated nucleic acid according to SEQ ID NO:1, SEQ ID NO:3, SEQ ID NO:5, SEQ ID NO:7, SEQ ID NO:9 or SEQ ID NO:11.

A further aspect of the invention refers to an expression system containing at least one gene according to SEQ ID NO:1, SEQ ID NO:3, SEQ ID NO:5, SEQ ID NO:7, SEQ ID NO:9 or SEQ ID NO:11, or up to 2, 3, 4, 5 or 6 genes according to SEQ ID NO: 1, SEQ ID NO:3, SEQ ID NO:5, SEQ ID NO:7, SEQ ID NO:9 or SEQ ID NO:11, or mixtures thereof.

A further aspect of the invention refers to a method of producing a variant strain as described above comprising the steps of
introducing into a cell of a parental fungus at least one gene of SEQ ID NO:1, SEQ ID NO:3, SEQ ID NO:5, SEQ ID NO:7, SEQ ID NO:9 or SEQ ID NO:11, and expressing said at least one gene in said cell.

A further aspect of the invention refers to a method of producing a variant strain as described above comprising the steps of
introducing into a cell of a parental fungus at least two, at least three, at least four, at least five or at least six gene of SEQ ID NO:1, SEQ ID NO:3, SEQ ID NO:5, SEQ ID NO:7, SEQ ID NO:9 or SEQ ID NO:11 and expressing said at genes in said cell.

A further aspect of the invention refers to the method as described above, wherein the filamentous fungus is Trichoderma reesei, specifically QM6a.

A further aspect of the invention refers to the method as described above, wherein the filamentous fungus comprises a gene encoding a heterologous protein.

A further aspect of the invention refers to a female fertile variant strain produced by a method as described above.

A further aspect of the invention refers to a heterologous protein produced by the female fertile variant strain as described above.

A further aspect of the invention refers to the method of producing a heterologous protein comprising the steps of cultivating the female fertile variant strain as described above and, optionally, recovering the heterologous protein.

The filamentous fungus Trichoderma reesei is further characterized by the biological material deposited at the DSMZ—Deutsche Sammlung von Mikroorganismen und Zellkulturen, Inhoffenstraβe 7B, 38124 Braunschweig (DE) under the accession numbers as indicated herein:
DSM 26183, Trichoderma reesei QMF1B (MAT1-1)
DSM 26184, Trichoderma reesei QMF2B (MAT1-2)
DSM 26185, Trichoderma reesei QMF1C (MAT1-1)
DSM 26186, Trichoderma reesei QMF2C (MAT1-2)
Deposition date: Jul. 23, 2012
Depositor: Technische Universität Wien, Vienna, Austria.

A further aspect of the invention refers to a filamentous fungus Trichoderma reesei deposited on Jul. 23, 2012 under Accession No. DSM 26183, DSM 26184, DSM 26185, or DSM 26186.

A further aspect of the invention refers to the use of the heterologous protein produced by the female fertile variant strain as described for use in industrial or pharmaceutical applications, preferably in food industry.

Further aspects of the invention relate to the introduction of one or more exogenous polynucleotide(s) encoding one or more mating polypeptide(s) into female sterile filamentous fungal cells, thereby converting the cell to a female fertile cell and, vice versa, to the inactivation of one or more native residing polynucleotide(s) encoding one or more mating polypeptide(s) in a female fertile fungal cell in order to render the cell sterile, as well as the resulting cells.

Accordingly, the invention relates to female fertile filamentous fungal cells comprising at least one exogenous polynucleotide encoding a mating polypeptide having an amino acid sequence at least 60% identical to a sequence chosen from the group of sequences consisting of SEQ ID NO:2, SEQ ID NO:4, SEQ ID NO:6, SEQ ID NO:8, SEQ ID NO:10 and SEQ ID NO:12; preferably the amino acid sequence is at least 65% identical, more preferably it is at least 65%, 70%, 75%, 80%, 85%, 90%, 95%, 97%, 98%, 99% or 100% identical to SEQ ID NO:2, SEQ ID NO:4, SEQ ID NO:6, SEQ ID NO:8, SEQ ID NO:10 or SEQ ID NO:12.

In addition, the invention relates to female fertile filamentous fungal cells comprising at least one exogenous polynucleotide, the cDNA of which having a nucleotide sequence at least 60% identical to the joined coding region(s) in SEQ ID NO:1, SEQ ID NO:3, SEQ ID NO:5, SEQ ID NO:7, SEQ ID NO:9 or SEQ ID NO:11; preferably the nucleotide sequence is at least 65%, 70%, 75%, 80%, 85%, 90%, 95%, 97%, 98%, 99% or 100% identical to the joined coding region(s) in SEQ ID NO:1, SEQ ID NO:3, SEQ ID NO:5, SEQ ID NO:7, SEQ ID NO:9 or SEQ ID NO:11.

Also, the invention relates to female fertile filamentous fungal cells comprising at least one exogenous polynucleotide having a nucleotide sequence at least 60% identical to SEQ ID NO:1, SEQ ID NO:3, SEQ ID NO:5, SEQ ID NO:7, SEQ ID NO:9 or SEQ ID NO:11; preferably the nucleotide sequence is at least 65%, 70%, 75%, 80%, 85%, 90%, 95%, 97%, 98%, 99% or 100% identical to SEQ ID NO:1, SEQ ID NO:3, SEQ ID NO:5, SEQ ID NO:7, SEQ ID NO:9 or SEQ ID NO:11.

Another aspect of the invention relates to female sterile filamentous fungal cells, wherein at least one polynucleotide encoding a mating polypeptide has been inactivated, said mating polypeptide comprising an amino acid sequence at least 60% identical to a sequence chosen from the group of sequences consisting of SEQ ID NO:2, SEQ ID NO:4, SEQ ID NO:6, SEQ ID NO:8, SEQ ID NO:10 and SEQ ID NO:12; preferably the amino acid sequence is at least 65% identical, more preferably it is at least 65%, 70%, 75%, 80%, 85%, 90%, 95%, 97%, 98%, 99% or 100% identical to SEQ ID NO:2, SEQ ID NO:4, SEQ ID NO:6, SEQ ID NO:8, SEQ ID NO:10 or SEQ ID NO:12.

Yet another aspect of the invention relates to female sterile filamentous fungal cells, wherein at least one polynucleotide encoding a mating polypeptide has been inactivated and said at least one polynucleotide has a nucleotide sequence at least 60% identical to the joined coding region(s) in SEQ ID NO:1, SEQ ID NO:3, SEQ ID NO:5, SEQ ID NO:7, SEQ ID NO:9 or SEQ ID NO:11; preferably the polynucleotide has a nucleotide sequence at least 65%, 70%, 75%, 80%, 85%, 90%, 95%, 97%, 98%, 99% or 100% identical to the joined coding region(s) in SEQ ID NO:1, SEQ ID NO:3, SEQ ID NO:5, SEQ ID NO:7, SEQ ID NO:9 or SEQ ID NO:11.

In addition, the invention relates to female sterile filamentous fungal cells, wherein at least one polynucleotide encoding a mating polypeptide has been inactivated and said at least one polynucleotide has a nucleotide sequence at least 60% identical to SEQ ID NO:1, SEQ ID NO:3, SEQ ID NO:5, SEQ ID NO:7, SEQ ID NO:9 or SEQ ID NO:11; preferably the polynucleotide has a nucleotide sequence at least 65%, 70%, 75%, 80%, 85%, 90%, 95%, 97%, 98%, 99% or 100% identical to SEQ ID NO:1, SEQ ID NO:3, SEQ ID NO:5, SEQ ID NO:7, SEQ ID NO:9 or SEQ ID NO:11.

Further, the invention relates to methods for converting a female sterile filamentous fungal cell to a female fertile cell, said method comprising a step of transforming the sterile cell with at least one polynucleotide encoding a mating polypeptide comprising an amino acid sequence at least 60% identical to a sequence chosen from the group of sequences consisting of SEQ ID NO:2, SEQ ID NO:4, SEQ ID NO:6, SEQ ID NO:8, SEQ ID NO:10 and SEQ ID NO:12, whereby the cell becomes fertile; preferably the amino acid sequence is at least 65% identical, more preferably it is at least 65%, 70%, 75%, 80%, 85%, 90%, 95%, 97%, 98%, 99% or 100% identical to SEQ ID NO:2, SEQ ID NO:4, SEQ ID NO:6, SEQ ID NO:8, SEQ ID NO:10 or SEQ ID NO:12.

An aspect of the invention relates to methods for converting a female sterile filamentous fungal cell to a female fertile cell, said method comprising a step of transforming the sterile cell with at least one polynucleotide encoding a mating polypeptide, wherein said at least one polynucleotide has a cDNA nucleotide sequence at least 60% identical to the joined coding region(s) in SEQ ID NO:1, SEQ ID NO:3, SEQ ID NO:5, SEQ ID NO:7, SEQ ID NO:9 or SEQ ID NO:11; preferably the nucleotide sequence is at least 65%, 70%, 75%, 80%, 85%, 90%, 95%, 97%, 98%, 99% or 100% identical to the joined coding region(s) in SEQ ID NO:1, SEQ ID NO:3, SEQ ID NO:5, SEQ ID NO:7, SEQ ID NO:9 or SEQ ID NO:11.

A method for converting a female sterile filamentous fungal cell to a female fertile cell, said method comprising a step of transforming the sterile cell with at least one polynucleotide encoding a mating polypeptide, wherein said at least one polynucleotide has a nucleotide sequence at least 60% identical to SEQ ID NO:1, SEQ ID NO:3, SEQ ID NO:5, SEQ ID NO:7, SEQ ID NO:9 or SEQ ID NO:11; preferably the nucleotide sequence is at least 65%, 70%, 75%, 80%, 85%, 90%, 95%, 97%, 98%, 99% or 100% identical to SEQ ID NO:1, SEQ ID NO:3, SEQ ID NO:5, SEQ ID NO:7, SEQ ID NO:9 or SEQ ID NO:11.

Yet another aspect of the invention relates to methods of converting a female fertile filamentous fungal cell to a female sterile cell, said method comprising the step of inactivating at least one polynucleotide encoding a mating polypeptide, wherein said mating polypeptide comprises an amino acid sequence at least 60% identical to a sequence chosen from the group of sequences consisting of SEQ ID NO:2, SEQ ID NO:4, SEQ ID NO:6, SEQ ID NO:8, SEQ ID NO:10 and SEQ ID NO:12; preferably the amino acid sequence is at least 65% identical, more preferably it is at least 65%, 70%, 75%, 80%, 85%, 90%, 95%, 97%, 98%, 99% or 100% identical to SEQ ID NO:2, SEQ ID NO:4, SEQ ID NO:6, SEQ ID NO:6, SEQ ID NO:8 or SEQ ID NO:12.

One more aspect of the invention relates to methods of converting a female fertile filamentous fungal cell to a female sterile cell, said method comprising the step of inactivating at least one polynucleotide encoding a mating polypeptide, wherein said polynucleotide has a cDNA nucleotide sequence at least 60% identical to the joined coding region(s) in SEQ ID NO:1, SEQ ID NO:3, SEQ ID NO:5, SEQ ID NO:7, SEQ ID NO:9 or SEQ ID NO:11; preferably the nucleotide sequence is at least 65%, 70%, 75%, 80%, 85%, 90%, 95%, 97%, 98%, 99% or 100% identical to the joined coding region(s) in SEQ ID NO:1, SEQ ID NO:3, SEQ ID NO:5, SEQ ID NO:7, SEQ ID NO:9 or SEQ ID NO:11.

Another aspect relates to methods of converting a female fertile filamentous fungal cell to a female sterile cell, said method comprising the step of inactivating at least one polynucleotide encoding a mating polypeptide, wherein said polynucleotide has a nucleotide sequence at least 60% identical to SEQ ID NO:1, SEQ ID NO:3, SEQ ID NO:5, SEQ ID NO:7, SEQ ID NO:9 or SEQ ID NO:11; preferably the nucleotide sequence is at least 65%, 70%, 75%, 80%, 85%, 90%, 95%, 97%, 98%, 99% or 100% identical to SEQ ID NO:1, SEQ ID NO:3, SEQ ID NO:5, SEQ ID NO:7, SEQ ID NO:9 or SEQ ID NO:11.

In a further aspect, the invention relates to methods of producing a polypeptide of interest, said method comprising cultivating a filamentous fungal host cell as defined in any of the other aspects of the invention under conditions conducive for the expression of the polypeptide; and, optionally recovering the polypeptide.

### BRIEF DESCRIPTION OF DRAWINGS

FIG. 1: Schematic representation of application of sexual crossing in strain improvement for available industrial production strains as well as optimization of strains bearing traits from nature isolates and their integration into suitable production strains. QMF stands for a female fertile derivative of QM6a of mating type MAT1-1 or MAT1-2 (see also below).

FIG. 2: Schematic representation of backcrossing of CBS999.97 MAT1-1 with QM6a to remove background introduced by crossing with CBS999.97.

FIG. 3: Mating of female fertile strains derived from QM6a. Strains of mating type MAT1-1 (QMF1x) are able to mate with those of mating type MAT1-2 (QMF2x), but not with themselves.

FIG. 4: Schematic representation of overlapping acquired regions in three strains. Recombination blocks are different in all three strains and are not constraint by repeat regions. This suggests that there is a region in the SNP interval (pink/blue) that is selected because it aids in fertility.

FIG. 5: Schematic representation of the outcome of a cross between female sterile QM6a and its female fertile derivative QMF1. Female fertile and sterile progeny appear in approximately equal numbers and were screened for the presence of the wild-type or mutated alleles.

### DEFINITIONS

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide having one or more (e.g., several) amino acids absent from the amino and/or carboxyl terminus of a mature polypeptide; wherein the fragment has retained the activity of the polypeptide.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Mature polypeptide: The term “mature polypeptide” means a polypeptide in its final form following translation and any post-translational modifications, such as N-terminal processing, C-terminal truncation, glycosylation, phosphorylation, etc.

Mature polypeptide coding sequence: The term “mature polypeptide coding sequence” means a polynucleotide that encodes a mature polypeptide.

Nucleic acid construct: The term “nucleic acid construct” means a nucleic acid molecule, either single or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Exogenous: The term “exogenous” herein means introduced from or produced outside of a cell. An exogenous polynucleotide in a cell, for example, has been introduced into the cell.

Female fertile or female sterile: Of course, filamentous fungal cells are neither male nor female in the usual meaning of the terms. However, in order for crossing to occur with a compatible partner, one of the two cells needs to be able to form so-called female reproductive organs or fruiting bodies. Accordingly, in the present context the terms “female fertile” or “female sterile” refer to the ability of a filamentous fungal host cell to form female reproductive organs, i.e. fruiting bodies, or not, upon crossing with a compatible partner.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970) as implemented in the Needle program of the EMBOSS package (EMBOSS: The European Molecular Biology Open Software Suite, Rice et al., 2000), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:
(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:
(Identical Deoxyribonucleotides×100)/(Length of Alignment−Total Number of Gaps in Alignment)

### Description of Embodiments

The present strains and methods relate to variant filamentous fungus cells having genetic modifications that affect their development, morphology and growth characteristics.

As used herein, the phrase “female fertile variant strain of filamentous fungus cells”, or similar phrases refer to fertile strains of filamentous fungus cells that are derived (i.e. obtained from or obtainable) from a female sterile parental strain belonging to filamentous fungus.

As used herein, the phrase “heterologous protein” refers to a polypeptide that is desired to be expressed in a filamentous fungus, optionally at high levels and for the purpose of commercialization. Such a protein may be an enzyme, a substrate-binding protein, a surface-active protein, a structural protein, or the like. The heterologous protein may be a hormone, enzyme, receptor or portion thereof, antibody or portion thereof, or reporter. For example, the protein may be an enzyme, such as, a hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

### Mating Polypeptides

A female specific mating polypeptide of the present invention preferably comprises or consists of an amino acid sequence which has at least 60% sequence identity to the sequence shown in SEQ ID NO:2, SEQ ID NO:4, SEQ ID NO:6, SEQ ID NO:8, SEQ ID NO:10 or SEQ ID NO:12; preferably at least 65% sequence identity, or at least 70%, 75%, 80%, 85%, 90%, 95%, 97%, 98%, 99% or 100% sequence identity. The mating polypeptide may also be an allelic variant thereof or a functional fragment thereof.

The polynucleotide of the female specific alleles shown in SEQ ID NO:1, SEQ ID NO:3, SEQ ID NO:5, SEQ ID NO:7, SEQ ID NO:9 or SEQ ID NO:11 or a subsequence thereof, as well as the polypeptide of SEQ ID NO: 2, SEQ ID NO:4, SEQ ID NO:6, SEQ ID NO:8, SEQ ID NO:10 or SEQ ID NO:12 or a functional fragment thereof, may be used to design nucleic acid probes to identify and clone female specific alleles encoding mating polypeptides from strains of different filamentous fungal genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for female specific allele DNA that hybridizes with the probes described above and encodes a mating polypeptide. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes, the carrier material is used in a Southern blot.

For purposes of the present invention, hybridization indicates that the polynucleotide hybridizes to a labeled nucleic acid probe corresponding to (i) SEQ ID NO:1; (ii) the mature polypeptide coding sequence of SEQ ID NO:1; (iii) the cDNA sequence thereof; (iv) the full-length complement thereof; or (v) a subsequence thereof; under very low to very high stringency conditions. Molecules to which the nucleic acid probe hybridizes under these conditions can be detected using, for example, X-ray film or any other detection means known in the art.

In another embodiment, the present invention relates to variants of the mature polypeptide of SEQ ID NO:2, SEQ ID NO:4, SEQ ID NO:6, SEQ ID NO:8, SEQ ID NO:10 or SEQ ID NO:12 comprising a substitution, deletion, and/or insertion at one or more (e.g., several) positions. In an embodiment, the number of amino acid substitutions, deletions and/or insertions introduced into the mature polypeptide of SEQ ID NO: 2, SEQ ID NO:4, SEQ ID NO:6, SEQ ID NO:8, SEQ ID NO:10 or SEQ ID NO:12 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill (1979). Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989). In the latter technique, single alanine mutations are introduced at every residue in the molecule, and the resultant mutant molecules are tested to identify amino acid residues that are critical to the activity of the molecule. See also, Hilton et al., (1996). The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer (1988); Bowie and Sauer (1989); WO 95/17413; or WO 95/22625. Other methods that can be used include error-prone PCR, phage display (e.g., Lowman et al. (1991); U.S. Pat. No. 5,223,409; WO 92/06204), and region-directed mutagenesis (Derbyshire et al. (1986)).

Mutagenesis/shuffling methods can be combined with high-throughput, automated screening methods to detect activity of cloned, mutagenized polypeptides expressed by host cells (Ness et al. (1999)). Mutagenized DNA molecules that encode active polypeptides can be recovered from the host cells and rapidly sequenced using standard methods in the art. These methods allow the rapid determination of the importance of individual amino acid residues in a polypeptide.

### Sources of Mating Polypeptides

A mating polypeptide of the present invention may be obtained from any filamentous fungal genus or species that has the capacity for sexual development. A preferred source is Candida, Kluyveromyces, Pichia, Saccharomyces, Schizosaccharomyces, or Yarrowia polypeptide; or a filamentous fungal polypeptide such as an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, lrpex, Lentinula, Leptospaeria, Magnaporthe, Melanocarpus, Meripilus, Mucor, Myceliophthora, Neocaffimastix, Neurospora, Paecilomyces, Peniciffium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria.

In another aspect, the polypeptide is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zona turn, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenaturn, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride polypeptide.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al. (1989)).

### Polynucleotides

The present invention also relates to isolated polynucleotides encoding a mating polypeptide of the present invention, as described herein. An isolated polynucleotide of the invention has at least 60% sequence identity to the nucleic acid sequence shown in SEQ ID NO:1, SEQ ID NO:3, SEQ ID NO:5, SEQ ID NO:7, SEQ ID NO:9 or SEQ ID NO:11; preferably at least 65% sequence identity, or at least 70%, 75%, 80%, 85%, 90%, 95%, 97%, 98%, 99% or 100% sequence identity.

The techniques used to isolate or clone a polynucleotide are known in the art and include isolation from genomic DNA or cDNA, or a combination thereof. The cloning of the polynucleotides from genomic DNA can be effected, e.g., by using the well-known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Trichoderma, or a related organism and thus, for example, may be an allelic or species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the polypeptide coding sequence of SEQ ID NO:1, SEQ ID NO:3, SEQ ID NO:5, SEQ ID NO:7, SEQ ID NO:9 or SEQ ID NO:11, e.g., a fragment or a subsequence thereof, and/or by introduction of nucleotide substitutions that do not result in a change in the amino acid sequence of the polypeptide, but which correspond to the codon usage of the intended host organism, or by introduction of nucleotide substitutions that may give rise to a different amino acid sequence. For a general description of nucleotide substitution, see, e.g., Ford et al. (1991).

The term “derivative” or “functionally active derivative” of a gene as used according to the invention herein means a sequence resulting from modification of the parent sequence by insertion, deletion or substitution of one or more amino acids or nucleotides within the sequence or at either or both of the distal ends of the sequence, and which modification does not affect (in particular impair) the activity of this sequence. In a preferred embodiment the functionally active derivative is
a) a biologically active fragment of the nucleotide sequence, the functionally active fragment comprising at least 50% of the sequence of the nucleotide sequence, preferably at least 60%, preferably at least 70%, more preferably at least 80%, still more preferably at least 90%, even more preferably at least 95% and most preferably at least 97%, 98% or 99%;
b) derived from the nucleotide sequence by at least one amino acid substitution, addition and/or deletion, wherein the functionally active derivative has a sequence identity to the nucleotide sequence or to the functionally active fragment as defined in a) of at least 50%, preferably at least 60%, preferably at least 70%, preferably at least 80%, still more preferably at least 90%, even more preferably at least 95% and most preferably at least 97%, 98% or 99%; and/or
c) consists of the nucleotide sequence and additionally at least one nucleotide heterologous to the nucleotide sequence, preferably wherein the functionally active derivative is derived from or identical to any of the naturally occurring variants of any of sequences of SEQ ID NO: 1, SEQ ID NO:3, SEQ ID NO:5, SEQ ID NO:7, SEQ ID NO:9 or SEQ ID NO:11.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Dania (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2 tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

The control sequence may also be a transcription terminator, which is recognized by a host cell to terminate transcription. The terminator is operably linked to the 3′ terminus of the polynucleotide encoding the polypeptide. Any terminator that is functional in the host cell may be used in the present invention.

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

The control sequence may also be an mRNA stabilizer region downstream of a promoter and upstream of the coding sequence of a gene which increases expression of the gene.

The control sequence may also be a leader, a non-translated region of an mRNA that is important for translation by the host cell. The leader is operably linked to the 5′ terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

The control sequence may also be a polyadenylation sequence, a sequence operably linked to the 3′ terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

The control sequence may also be a signal peptide coding region that encodes a signal peptide linked to the N terminus of a polypeptide and directs the polypeptide into the cell's secretory pathway. The 5′ end of the coding sequence of the polynucleotide may inherently contain a signal peptide coding sequence naturally linked in translation reading frame with the segment of the coding sequence that encodes the polypeptide. Alternatively, the 5′ end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

The control sequence may also be a propeptide coding sequence that encodes a propeptide positioned at the N terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N terminus of a polypeptide and the signal peptide sequence is positioned next to the N terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′ phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of origins of replication useful in a filamentous fungal cell are AMA1 and ANS1 (Gems et al. (1991), Cullen et al. (1987); WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al. (1989)). Host cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be a filamentous fungal cell. “Filamentous fungi” include all filamentous forms of the subdivision Eumycota and Oomycota (as defined by Hawksworth et al., 1995). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium Mops, Chrysosporium keratinophilum, Chrysosporium lucknowense, Chrysosporium merdarium, Chrysosporium pannicola, Chrysosporium queenslandicum, Chrysosporium tropicum, Chrysosporium zona turn, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticula turn, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., (1984) and Christensen et al., (1988). Suitable methods for transforming Fusarium species are described by Malardier et al., (1989) and WO 96/00787.

Development of T.reesei

Sexual development of T. reesei was achieved only recently and defined this fungus as heterothallic (Seidl et al, 2009). Since QM6a was the only strain with widespread use of T. reesei, and hence only one mating type was available, a wild-type isolate (CBS999.97) of opposite mating type was used as crossing partner. Analysis at the molecular level revealed that QM6a is of mating type MAT1-2 and therefore requires a strain of MAT1-1 as mating partner. In order to be able to cross different production strains of T. reesei derived from QM6a, the genomic area comprising the MAT1-1 idiomorph of CBS999.97 was introduced into QM6a, which then should result in the capability to undergo sexual development with MAT1-2 strains. However, although this strategy was successful for the wild-type isolate CBS999.97 (mating type could be artificially switched), the derivative of QM6a, then containing the other mating type, could not be crossed with QM6a, which was interpreted as female sterility or lack of the ability to produce female reproductive structures (Seidl et al, 2009).

Schuster et al. (2012) disclose a method for obtaining a female fertile variant (QF1) of Trichoderma reesei QM9414. QM9414 represents a classical mutant strain obtained by random mutagenesis of the wild-type isolate QM6a, which has considerably increased cellulase production. Consequently, this strain shares the same defect in female fertility as QM6a. Female sterile QM9414 is crossed with female fertile CBS999.97 and QF1 is obtained by repeated backcrossing of female fertile segregates with QM9414. In that study, no reference is made to the molecular determinants (presence, regulation or sequence of genes) required for female fertility in QM414 or QM6a and also no experiments that would provide any information on such genes was reported. In contrast, the present invention discloses the six genes which are responsible for the female fertility, irrespective of the Trichoderma strain.

Although crossing T. reesei has become possible, the female sterility of QM6a and all its progeny used in research and industry represents a serious drawback for application of this tool for strain improvement. Currently, crossing of strains derived from QM6a is only possible with female fertile (wild-type) isolates, which often have a considerably different phenotype from QM6a. Such a cross is likely to lead to unpredictable phenotypes of progeny and a very high screening effort to evaluate the retention of the desired characteristic, due to integration of the genomic content of the wild-type isolate.

Consequently, strains bearing the QM6a genomic background, but which are female fertile would be necessary to keep introduction of foreign genomic content at a minimum. The present invention provides for the first time the six alleles which originate from CBS999.97 in QM6a derivatives which correlate with female fertility of the respective strains.

With the backcrossed strains we provide important tools for crossing of industrial high performance strains. These strains are mostly conventional mutants, which during numerous mutation cycles (using radiation and mutagenic chemicals) not only acquired mutations beneficial for cellulase gene expression or heterologous protein production, but also suffered mutations deleterious to growth and fitness. Such undesired mutations can be removed by crossing and selection. The mentioned backcrossed stains are advantageous in this respect, because their phenotype is much closer to that of the industrial strains (QM6a background) than to that of CBS999.97, which lowers the danger of a too dominant wild-type background.

Knowledge on the defect of QM6a causing female sterility can be used to check for fertility of progeny from crosses of a fertile QM6a derivative with industrial production strains, because the presence/absence of original or mutated alleles of the respective gene(s) would be indicative of female fertility. Using this approach it is possible to remove undesired mutations decreasing growth and/or fitness of production strains while keeping their beneficial characteristics in terms of enzyme production and substrate specificity. At the same time checking for genes reflecting introduction of female fertility genes results in strains ready for further crosses and straight forward improvement.

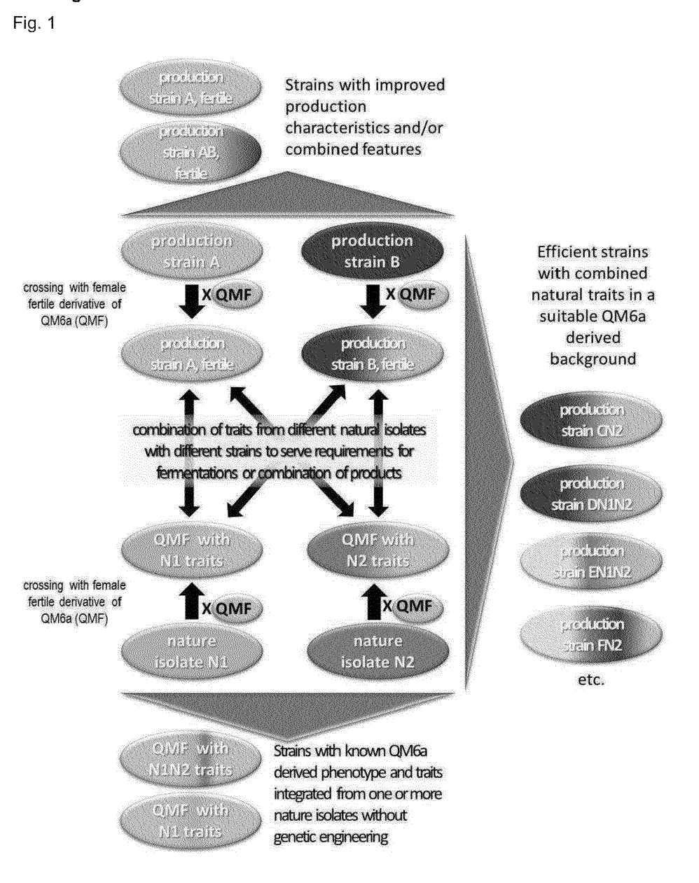

The possibility to check progeny for mating type will add to this benefit and enable selection of fertile progeny of suitable mating type for further crosses with other production strains bearing different characteristics or with the parental production strain to decrease QM6a background despite retaining fertility and reinforcing beneficial conditions (FIG. 1).

Since such strains will have been constructed using natural tools, which evolved with the fungi (sexual development instead of genetic engineering or use of mutagenic chemicals or radiation), use of enzymes and metabolites of strains will be safe even for use in the food industry and for pharmaceuticals (FIG. 1).

Female fertile backcrossed strains derived from QM6a could be used for crossing with nature isolates producing novel compounds or enzymes even if these wild-type isolates are female sterile (as is QM6a) or if the nature isolate bearing the desired characteristics happens to be of mating type MAT1-2, which cannot be crossed to QM6a right away. It should be noted here that female sterility is not uncommon in nature, since asexual reproduction is less resource consuming than sexual reproduction, which provides the female sterile part of a population with a certain evolutionary advantage (Taylor et al., 1999).

Progeny of crosses with nature isolates could be screened for production of this compound/enzyme and (if necessary after several rounds of backcrossing), a strain showing the well-known characteristics of T. reesei QM6a for industrial use in fermentations as well as production of the novel enzyme or compound. Using the female fertile strains derived from QM6a in this process will provide a significant advantage in breeding, since after every cycle, progeny would be screened and the best candidate for further improvement could appear in both mating types. Having fertile strains of both mating types with QM6a background available will be highly beneficial for strain improvement using this strategy.

The same of course applies for strain improvement with currently already available (female sterile) production strains using sexual development for conventional breeding. With crossing and if desired, several rounds of backcrossing or crossing with other production strains and intermittent screening for improved characteristics, significant strain improvement and flexibility in adjusting strains to production conditions can be achieved.

Also, using sexual development with female fertile strains derived from QM6a can be used to first enhance the desired characteristic acquired from a nature isolate, albeit if the genes responsible for production of the characteristic/compound are not known, and thereafter this characteristic can be integrated into an available production strain. Again, knowledge on genes responsible for female fertility in the strains derived from QM6a can be used to make sure that the ability to undergo sexual development will not be lost during repeated crossing cycles.

Besides the possibility to check progeny from crosses with female fertile derivatives of QM6a, in order to retain their female fertility after crossing with female sterile production strains, knowledge on the defect could be used to alleviate any inability to undergo sexual development in a strain.

Creating double mutants by crossing requires much less hands on time and is much more efficient than using the conventional method of transformation (the probability of creating a double mutant is around 25%). Also here, our new strains with the genomic background of QM6a, but the ability to undergo sexual development, significantly contributes to this process. Thereby methods used in other model fungi such as Aspergillus nidulans or Neurospora crassa for decades now also become feasible for T. reesei. Especially in the important area of biofuels research, this contribution will be significant.

Considering the high number of industrially applied fungi for which sexual development has not yet been achieved, the data provided on the genomic regions responsible for the sexual defect of T. reesei can also be applied to these fungi, which may provide a basis for engineering them to enable sexual development.

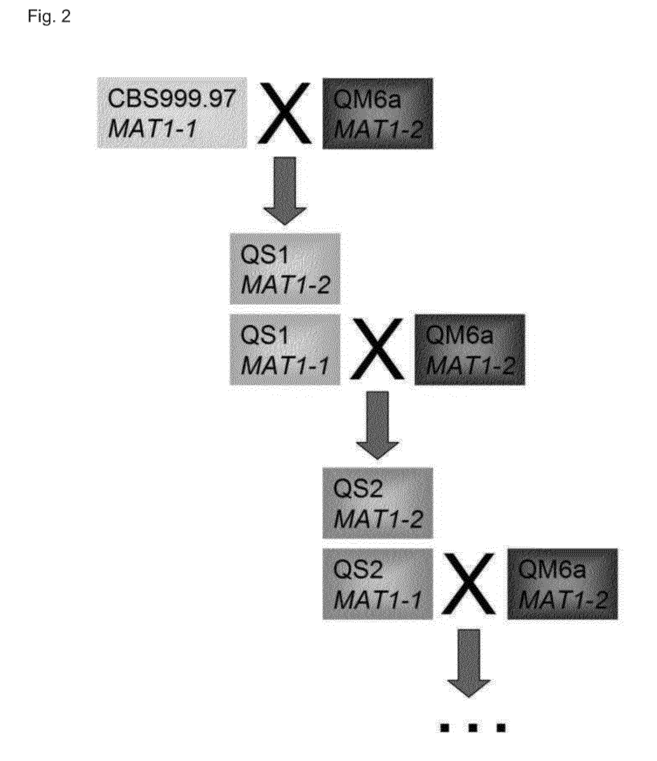

The aim of this project was to gain deeper insights into the process of sexual development in Trichoderma reesei/Hypocrea jecorina. Thereby the molecular basis for the most important issue, female sterility of the parental strain of all strains used in research and industry, QM6a, was elucidated. This was accomplished by the construction of a female fertile strain by crossing of QM6a (MAT1-2) with the sexually competent wild-type isolate CBS999.97 (MAT1-1) (Seidl et al, 2009) and subsequent screening for mating type, phenotype and sexual competence. Strains of mating type MAT1-1, which had gained female fertility after this cross were backcrossed with QM6a and again screened for required features. In total ten rounds of crossing, ascospore isolation and screening were performed (FIG. 2).

This strategy was meant to create numerous female fertile strains which had regained the capability to undergo sexual development even with a female sterile mating partner (for example an industrial production strain derived from female sterile QM6a or its derivatives).

Sequencing of several of the resulting strains allowed for delineation of the genomic area(s), which were indeed crucial for female fertility of the backcrossed strains derived from QM6a. Analysis of 54 additional strains from independent subsequent crosses (confirmed female fertile and female sterile strains of MAT1-1 and MAT1-2) proved the relevance of 6 genes for female fertility. Knowledge on the gene(s) within this area allows for rapid checking of fertility of progeny from crosses. Also, introduction of one or more gene(s) from this area leads to female fertility of strains derived from QM6a, if the previous mutation cycles have not resulted in loss of other genes essential for sexual development.

The present strains and methods are useful in the production of commercially important proteins including, for example, cellulases, xylanases, pectinases, proteases, amylases, pullunases, lipases, esterases, perhydrolases, transferases, laccases, catalases, oxidases, reductases, hydrophobin and other enzymes and non-enzyme proteins capable of being expressed in filamentous fungi. Such proteins may be for industrial or pharmaceutical use.

### Removal or Reduction of Fertility

The present invention also relates to methods of producing a mutant of a parent cell, which comprises disrupting or deleting one or more polynucleotide according to the invention as shown in SEQ ID NO:1, SEQ ID NO:3, SEQ ID NO:5, SEQ ID NO:7, SEQ ID NO:9 or SEQ ID NO:11, that is involved in fertility or mating ability in a filamentous fungal cell, or a portion thereof, encoding one or more polypeptide, respectively, that is involved in fertility or mating ability as shown in SEQ ID NO:2, SEQ ID NO:4, SEQ ID NO:6, SEQ ID NO:8, SEQ ID NO:10 or SEQ ID NO:12, which results in the mutant cell producing less or even nothing at all of the polypeptide than the parent cell when cultivated under the same conditions.

The non-fertile or sterile mutant cell may be constructed by reducing or eliminating expression of the polynucleotide using methods well known in the art, for example, insertions, disruptions, replacements, or deletions. In a preferred aspect, the polynucleotide is inactivated. The polynucleotide to be modified or inactivated may be, for example, the coding region or a part thereof essential for activity, or a regulatory element required for expression of the coding region. An example of such a regulatory or control sequence may be a promoter sequence or a functional part thereof, i.e., a part that is sufficient for affecting expression of the polynucleotide. Other control sequences for possible modification include, but are not limited to, a leader, polyadenylation sequence, propeptide sequence, signal peptide sequence, transcription terminator, and transcriptional activator.

Modification or inactivation of the polynucleotide may be performed by subjecting the parent cell to mutagenesis and selecting for mutant cells in which expression of the polynucleotide has been reduced or eliminated. The mutagenesis, which may be specific or random, may be performed, for example, by use of a suitable physical or chemical mutagenizing agent, by use of a suitable oligonucleotide, or by subjecting the DNA sequence to PCR generated mutagenesis. Furthermore, the mutagenesis may be performed by use of any combination of these mutagenizing agents.

Examples of a physical or chemical mutagenizing agent suitable for the present purpose include ultraviolet (UV) irradiation, hydroxylamine, N methyl-N′ nitro-N nitrosoguanidine (MNNG), O methyl hydroxylamine, nitrous acid, ethyl methane sulphonate (EMS), sodium bisulphite, formic acid, and nucleotide analogues.

When such agents are used, the mutagenesis is typically performed by incubating the parent cell to be mutagenized in the presence of the mutagenizing agent of choice under suitable conditions, and screening and/or selecting for mutant cells exhibiting reduced or no expression of the gene.

Modification or inactivation of the polynucleotide may be accomplished by insertion, substitution, or deletion of one or more nucleotides in the gene or a regulatory element required for transcription or translation thereof. For example, nucleotides may be inserted or removed so as to result in the introduction of a stop codon, the removal of the start codon, or a change in the open reading frame. Such modification or inactivation may be accomplished by site-directed mutagenesis or PCR generated mutagenesis in accordance with methods known in the art. Although, in principle, the modification may be performed in vivo, i.e., directly on the cell expressing the polynucleotide to be modified, it is preferred that the modification be performed in vitro as exemplified below.

An example of a convenient way to eliminate or reduce expression of a polynucleotide is based on techniques of gene replacement, gene deletion, or gene disruption. For example, in the gene disruption method, a nucleic acid sequence corresponding to the endogenous polynucleotide is mutagenized in vitro to produce a defective nucleic acid sequence that is then transformed into the parent cell to produce a defective gene. By homologous recombination, the defective nucleic acid sequence replaces the endogenous polynucleotide. It may be desirable that the defective polynucleotide also encodes a marker that may be used for selection of transformants in which the polynucleotide has been modified or destroyed. In an aspect, the polynucleotide is disrupted with a selectable marker such as those described herein.

The present invention also relates to methods of inhibiting the expression of a polypeptide having [enzyme] activity in a cell, comprising administering to the cell or expressing in the cell a double-stranded RNA (dsRNA) molecule, wherein the dsRNA comprises a subsequence of a polynucleotide of the present invention. In a preferred aspect, the dsRNA is about 15, 16, 17, 18, 19, 20, 21, 22, 23, 24, 25 or more duplex nucleotides in length.

The dsRNA is preferably a small interfering RNA (siRNA) or a micro RNA (miRNA). In a preferred aspect, the dsRNA is small interfering RNA for inhibiting transcription. In another preferred aspect, the dsRNA is micro RNA for inhibiting translation.

The present invention also relates to such double-stranded RNA (dsRNA) molecules, comprising a portion of the mature polypeptide coding sequence of SEQ ID NO: 1, SEQ ID NO:3, SEQ ID NO:5, SEQ ID NO:7, SEQ ID NO:9 or SEQ ID NO:11, for inhibiting expression of the polypeptide in a cell. While the present invention is not limited by any particular mechanism of action, the dsRNA can enter a cell and cause the degradation of a single-stranded RNA (ssRNA) of similar or identical sequences, including endogenous mRNAs. When a cell is exposed to dsRNA, mRNA from the homologous gene is selectively degraded by a process called RNA interference (RNAi).

The dsRNAs of the present invention can be used in gene-silencing. In one aspect, the invention provides methods to selectively degrade RNA using a dsRNAi of the present invention. The process may be practiced in vitro, ex vivo or in vivo. In one aspect, the dsRNA molecules can be used to generate a loss-of-function mutation in a cell, an organ or an animal. Methods for making and using dsRNA molecules to selectively degrade RNA are well known in the art; see, for example, U.S. Pat. Nos. 6,489,127; 6,506,559; 6,511,824; and 6,515,109.

The present invention further relates to a mutant cell of a parent cell that comprises a disruption or deletion of a polynucleotide encoding the polypeptide or a control sequence thereof or a silenced gene encoding the polypeptide, which results in the mutant cell producing less of the polypeptide or no polypeptide compared to the parent cell.

The polypeptide-deficient mutant cells are particularly useful as host cells for expression of native and heterologous polypeptides. Therefore, the present invention further relates to methods of producing a native or heterologous polypeptide, comprising (a) cultivating the mutant cell under conditions conducive for production of a native or heterologous polypeptide of interest, especially an enzyme of interest; and (b) recovering the polypeptide or enzyme. The term “heterologous polypeptides” means polypeptides that are not native to the host cell, e.g., a variant of a native protein. The host cell may comprise more than one copy of a polynucleotide encoding the native or heterologous polypeptide.

The methods used for cultivation and purification of the product of interest may be performed by methods known in the art.

### Methods of Production

The present invention also relates to methods of producing a heterologous polypeptide, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Fermentation Broth Formulations or Cell Compositions

The present invention also relates to a fermentation broth formulation or a cell composition comprising a polypeptide of the present invention. The fermentation broth product further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4 methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

The invention is further defined in the following paragraphs:
1. A female fertile filamentous fungal cell comprising at least one exogenous polynucleotide encoding a mating polypeptide having an amino acid sequence at least 60% identical to a sequence chosen from the group of sequences consisting of SEQ ID NO:2, SEQ ID NO:4, SEQ ID NO:6, SEQ ID NO:8, SEQ ID NO:10 and SEQ ID NO:12; preferably the amino acid sequence is at least 65% identical, more preferably it is at least 65%, 70%, 75%, 80%, 85%, 90%, 95%, 97%, 98%, 99% or 100% identical to SEQ ID NO:2, SEQ ID NO:4, SEQ ID NO:6, SEQ ID NO:8, SEQ ID NO:10 or SEQ ID NO:12.
2. A female fertile filamentous fungal cell comprising at least one exogenous polynucleotide, the cDNA of which having a nucleotide sequence at least 60% identical to the joined coding region(s) in SEQ ID NO:1, SEQ ID NO:3, SEQ ID NO:5, SEQ ID NO:7, SEQ ID NO:9 or SEQ ID NO:11; preferably the nucleotide sequence is at least 65%, 70%, 75%, 80%, 85%, 90%, 95%, 97%, 98%, 99% or 100% identical to the joined coding region(s) in SEQ ID NO:1, SEQ ID NO:3, SEQ ID NO:5, SEQ ID NO:7, SEQ ID NO:9 or SEQ ID NO:11.
3. A female fertile filamentous fungal cell comprising at least one exogenous polynucleotide having a nucleotide sequence at least 60% identical to SEQ ID NO:1, SEQ ID NO:3, SEQ ID NO:5, SEQ ID NO:7, SEQ ID NO:9 or SEQ ID NO:11; preferably the nucleotide sequence is at least 65%, 70%, 75%, 80%, 85%, 90%, 95%, 97%, 98%, 99% or 100% identical to SEQ ID NO:1, SEQ ID NO:3, SEQ ID NO:5, SEQ ID NO:7, SEQ ID NO:9 or SEQ ID NO:11.
4. A female sterile filamentous fungal cell, wherein at least one exogenous polynucleotide encoding a mating polypeptide has been inactivated, said mating polypeptide comprising an amino acid sequence at least 60% identical to a sequence chosen from the group of sequences consisting of SEQ ID NO:2, SEQ ID NO:4, SEQ ID NO:6, SEQ ID NO:2, SEQ ID NO:4 and SEQ ID NO:6; preferably the amino acid sequence is at least 65% identical, more preferably it is at least 65%, 70%, 75%, 80%, 85%, 90%, 95%, 97%, 98%, 99% or 100% identical to SEQ ID NO:2, SEQ ID NO:4, SEQ ID NO:6, SEQ ID NO:2, SEQ ID NO:4 or SEQ ID NO:6.
5. A female sterile filamentous fungal cell, wherein at least one exogenous polynucleotide encoding a mating polypeptide has been inactivated and said at least one polynucleotide has a nucleotide sequence at least 60% identical to the joined coding region(s) in SEQ ID NO:1, SEQ ID NO:3, SEQ ID NO:5, SEQ ID NO:7, SEQ ID NO:9 or SEQ ID NO:11; preferably the polynucleotide has a nucleotide sequence at least 65%, 70%, 75%, 80%, 85%, 90%, 95%, 97%, 98%, 99% or 100% identical to the joined coding region(s) in SEQ ID NO:1, SEQ ID NO:3, SEQ ID NO:5, SEQ ID NO:7, SEQ ID NO:9 or SEQ ID NO:11.
6. A female sterile filamentous fungal cell, wherein at least one exogenous polynucleotide encoding a mating polypeptide has been inactivated and said at least one polynucleotide has a nucleotide sequence at least 60% identical to SEQ ID NO:1, SEQ ID NO:3, SEQ ID NO:5, SEQ ID NO:7, SEQ ID NO:9 or SEQ ID NO:11; preferably the polynucleotide has a nucleotide sequence at least 65%, 70%, 75%, 80%, 85%, 90%, 95%, 97%, 98%, 99% or 100% identical to SEQ ID NO:1, SEQ ID NO:3, SEQ ID NO:5, SEQ ID NO:7, SEQ ID NO:9 or SEQ ID NO:11.
7. The filamentous fungal cell of any preceding paragraph which is an Acremonium, Agaricus, Altemaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberlla, Holomastigotoides, Humicola, lrpex, Lentinula, Leptospaeria, Magnaporthe, Melanocarpus, Meripilus, Mucor, Myceliophthora, Neocallimastix, Neurospora, Paecilomyces, Peniciffium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria cell; preferably an Aspergillus or Trichoderma cell.
8. The filamentous fungal cell of paragraph 7 which is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zona turn, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride cell; preferably an Aspergillus niger, Aspergillus oryzae or Trichoderma reesei cell.
9. The filamentous fungal cell of any preceding paragraph which comprises a polynucleotide encoding a polypeptide of interest, preferably the polypeptide of interest is a hormone, enzyme, receptor or portion thereof, antibody or portion thereof, or reporter; more preferably the polypeptide of interest is a hydrolase, isomerase, ligase, lyase, oxidoreductase, or transferase enzyme; most preferably the polypeptide of interest is an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase
10. The filamentous fungal cell of paragraph 9, wherein the polynucleotide encoding the polypeptide of interest is exogenous or endogenous to the cell.
11. The filamentous fungal cell of paragraph 9 or 10, wherein the polynucleotide encoding the polypeptide of interest is present in the cell in two or more copies; preferably the two or more copies are integrated into the chromosome of the cell.
12. A method for converting a female sterile filamentous fungal cell to a fertile cell, said method comprising a step of transforming the sterile cell with at least one polynucleotide encoding a mating polypeptide comprising an amino acid sequence at least 60% identical to a sequence chosen from the group of sequences consisting of SEQ ID NO:2, SEQ ID NO:4, SEQ ID NO:6, SEQ ID NO:8, SEQ ID NO:10 and SEQ ID NO:12, whereby the cell becomes fertile; preferably the amino acid sequence is at least 65% identical, more preferably it is at least 65%, 70%, 75%, 80%, 85%, 90%, 95%, 97%, 98%, 99% or 100% identical to SEQ ID NO:2, SEQ ID NO:4, SEQ ID NO:6, SEQ ID NO:8, SEQ ID NO:10 or SEQ ID NO:12.
13. A method for converting a female sterile filamentous fungal cell to a fertile cell, said method comprising a step of transforming the sterile cell with at least one polynucleotide encoding a mating polypeptide, wherein said at least one polynucleotide has a cDNA nucleotide sequence at least 60% identical to the joined coding region(s) in SEQ ID NO:1, SEQ ID NO:3, SEQ ID NO:5, SEQ ID NO:7, SEQ ID NO:9 or SEQ ID NO:11; preferably the nucleotide sequence is at least 65%, 70%, 75%, 80%, 85%, 90%, 95%, 97%, 98%, 99% or 100% identical to the joined coding region(s) in SEQ ID NO:1, SEQ ID NO:3, SEQ ID NO:5, SEQ ID NO:7, SEQ ID NO:9 or SEQ ID NO:11.
14. A method for converting a female sterile filamentous fungal cell to a fertile cell, said method comprising a step of transforming the sterile cell with at least one polynucleotide encoding a mating polypeptide, wherein said at least one polynucleotide has a nucleotide sequence at least 60% identical to SEQ ID NO:1, SEQ ID NO:3, SEQ ID NO:5, SEQ ID NO:7, SEQ ID NO:9 or SEQ ID NO:11; preferably the nucleotide sequence is at least 65%, 70%, 75%, 80%, 85%, 90%, 95%, 97%, 98%, 99% or 100% identical to SEQ ID NO:1, SEQ ID NO:3, SEQ ID NO:5, SEQ ID NO:7, SEQ ID NO:9 or SEQ ID NO:11.
15. A method of converting a female fertile filamentous fungal cell to a sterile cell, said method comprising the step of inactivating at least one polynucleotide encoding a mating polypeptide, wherein said mating polypeptide comprises an amino acid sequence at least 60% identical to a sequence chosen from the group of sequences consisting of SEQ ID NO:2, SEQ ID NO:4, SEQ ID NO:6, SEQ ID NO:2, SEQ ID NO:4 and SEQ ID NO:6; preferably the amino acid sequence is at least 65% identical, more preferably it is at least 65%, 70%, 75%, 80%, 85%, 90%, 95%, 97%, 98%, 99% or 100% identical to SEQ ID NO:2, SEQ ID NO:4, SEQ ID NO:6, SEQ ID NO:2, SEQ ID NO:4 or SEQ ID NO:6.
16. A method of converting a female fertile filamentous fungal cell to a sterile cell, said method comprising the step of inactivating at least one polynucleotide encoding a mating polypeptide, wherein said polynucleotide has a cDNA nucleotide sequence at least 60% identical to the joined coding region(s) in SEQ ID NO:1, SEQ ID NO:3, SEQ ID NO:5, SEQ ID NO:7, SEQ ID NO:9 or SEQ ID NO:11; preferably the nucleotide sequence is at least 65%, 70%, 75%, 80%, 85%, 90%, 95%, 97%, 98%, 99% or 100% identical to the joined coding region(s) in SEQ ID NO:1, SEQ ID NO:3, SEQ ID NO:5, SEQ ID NO:7, SEQ ID NO:9 or SEQ ID NO:11.
17. A method of converting a female fertile filamentous fungal cell to a sterile cell, said method comprising the step of inactivating at least one polynucleotide encoding a mating polypeptide, wherein said polynucleotide has a nucleotide sequence at least 60% identical to SEQ ID NO:1, SEQ ID NO:3, SEQ ID NO:5, SEQ ID NO:7, SEQ ID NO:9 or SEQ ID NO:11; preferably the nucleotide sequence is at least 65%, 70%, 75%, 80%, 85%, 90%, 95%, 97%, 98%, 99% or 100% identical to SEQ ID NO:1, SEQ ID NO:3, SEQ ID NO:5, SEQ ID NO:7, SEQ ID NO:9 or SEQ ID NO:11.
18. The method of any of paragraphs 12 to 17, wherein the host cell comprises a polynucleotide encoding a polypeptide of interest; preferably the polypeptide of interest is a hormone, enzyme, receptor or portion thereof, antibody or portion thereof, or reporter; more preferably the polypeptide of interest is a hydrolase, isomerase, ligase, lyase, oxidoreductase, or transferase enzyme; most preferably the polypeptide of interest is an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.
19. The method of paragraph 18, wherein the polynucleotide encoding the polypeptide of interest is exogenous or endogenous to the cell.
20. The method of paragraph 18 or 19, wherein the polynucleotide encoding the polypeptide of interest is present in the cell in two or more copies; preferably the two or more copies are integrated into the chromosome of the cell.
21. A method of producing a polypeptide of interest, said method comprising cultivating a filamentous fungal host cell as defined in any of paragraphs 1 to 11 under conditions conducive for the expression of the polypeptide; and, optionally recovering the polypeptide.

### EXAMPLES

The Examples which follow are set forth to aid in the understanding of the invention but are not intended to, and should not be construed to limit the scope of the invention in any way. The Examples do not include detailed descriptions of conventional methods, e.g., cloning, transfection, and basic aspects of methods for overexpressing proteins in microbial host cells. Such methods are well known to those of ordinary skill in the art.

Production of Trichoderma reesei Strains by Non-Transgenic Methods

After screening of a wild-type isolate of Trichoderma reesei for production of an enzyme of interest or the ability to degrade as substrate of interest, this isolate can be crossed with the suitable mating type of the female fertile strains derived from QM6a as described above. Screening of progeny from this sexual cross will show which of these strains have retained the desired characteristic. After selection of well performing strains of both mating types, crossing can be repeated in order to enhance the efficiency of the strains in producing the desired enzyme or substrate degradation competence. Conventional breeding can be performed using this approach and the efficiency of QM6a in fermentation along with the extensive technological knowledge in application of this strain will be valuable for developing an efficient production process for enzymes of metabolic capabilities derived from natural isolates.

Construction of Trichoderma reesei Strains for Investigation of Female Fertility

Backcrossing of strains as described above was performed for 10 rounds to remove background of the wild-type isolate CBS999.97 which is not essential for female fertility in the resulting QM6a derivatives. Since the phenotype of CBS999.97 is different from that of QM6a (Seidl et al, 2009), it is desirable to use only strains closely related to production strains, i.e. derived from QM6a as crossing partner.

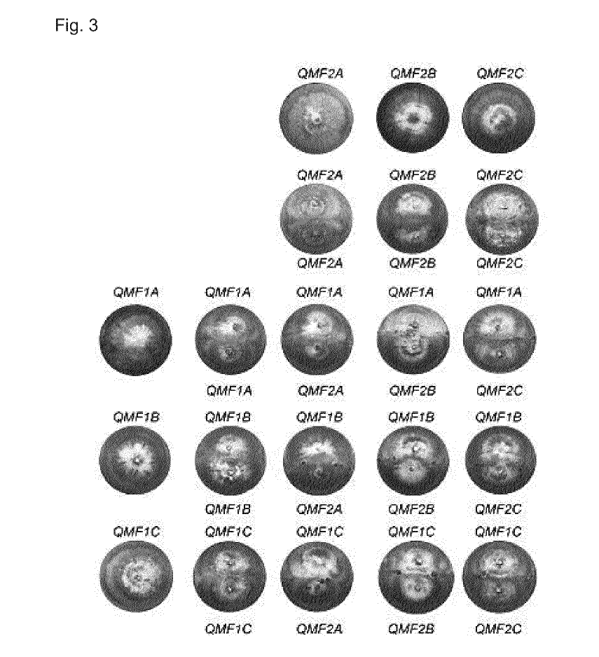

Numerous positive strains have been obtained, which were all finally checked and retested for mating type and female fertility. Mating analysis revealed successful construction of female fertile derivatives of QM6a (FIG. 3) with mating type MAT1-1 (denominated QMF1x) and mating type MAT1-2 (denominated QMF2x).

### Genome Analysis of Sexually Competent Derivatives of QM6a

For genome analysis of sexually competent derivatives of QM6a, three strains resulting from different, independent lines of crossing were chosen in order to ensure independent heritage of genomic loci responsible for re-introduction of female fertility. These three different strains correspond to QMF1A, QMF1B and QMF1C.

Sequencing these three strains was performed by next generation sequencing. Sequence assembly was performed by modelling to the available genome sequence of QM6a (http://genome.jgi-psf.org/Trire2/Trire2.home.html) and the other publicly available genome sequences of Trichoderma atroviride and Trichoderma virens (http://genome.jgi-psf.org).

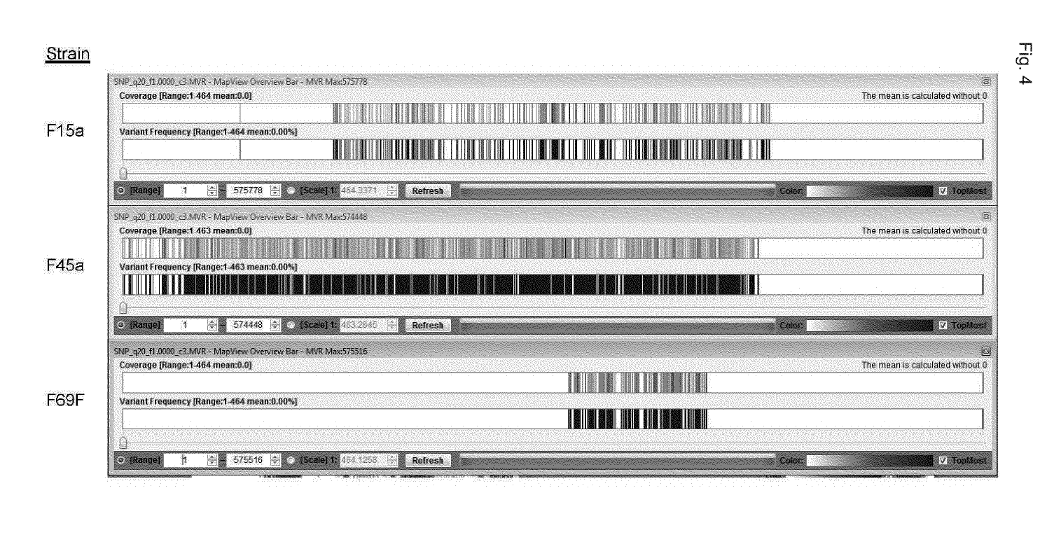

The genome of QMF1A, QMF1B and QMF1C revealed clear sequences acquired from CBS999.97 as reflected by an increased abundance of SNPs (single nucleotide polymorphisms) in the respective areas and consequently likely to be responsible for gaining the ability to mate by these strains. Comparing the three genomes resulted in overlapping genomic areas, which are assumed to contain the genes responsible for female fertility (FIG. 4). In total three different genomic regions on different scaffolds were identified to be consistently retained in these strains, which comprise almost 100 genes bearing mutations compared to QM6a.

The analysis showed no complete deletions in genes, but mostly point mutations and smaller alterations. From these regions narrowed down in the most likely genomic area, and the genes in this area along with several more genes from other genomic regions to be used as negative controls were used for further analysis.

Correlation Analysis

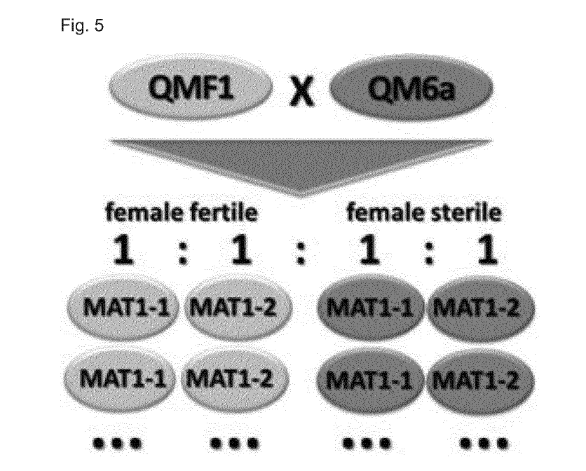

In order to be able to further delineate the genes responsible for the mating defect of QM6a, an additional cross between QM6a and QMF1A, QMF1 B and QMF1 C was performed. In total, 100 progeny of these crosses were isolated and tested for female fertility and mating type. About 20 female fertile and 20 female sterile strains were selected for further analysis (FIG. 5).

PCR-primer pairs were designed specific for amplification of the genomic sequence of the QM6a allele or the CBS999.97 allele (the wild-isolate), respectively. Using these primers enabled to distinguish, whether the strain would have retained the QM6a specific gene or acquired the CBS999.97 specific genes present in QMF1A, B and C.

Within two of the three possible genomic regions, this analysis revealed more or less random distribution. However, in one region up to 90% of correlation (p-values 1 e-07 for female fertility) between the presence of the CBS999.97 allele of the respective genes and female and male fertility in this strain was found. It was concluded that the six genes, for which this characteristic is valid, are responsible for the sexual defect in QM6a.

These genes are (protein IDs of the JGI T. reesei Genome Database, v2.0):
TR_80941 (FS_4)
TR_67350 (FS_5)
TR_51197 (FS_6)
TR_123422 (FS_7)
TR_80956 (FS_8)
TR_67418 (FS_9)

The same analysis with 14 additional strains of the respective opposite mating type confirmed this result and showed that the effect in QM6a is not mating-type dependent.

### Material and Methods

### Strains and Cultivation Conditions

T. reesei QM6a (ATCC 13631) and Hypocrea jecorina CBS999.97 (MAT1-1) (Seidl et al, 2009) were used. QM6a, CBS999.97 and all strains derived from them were maintained on 3% (w/v) malt extract with 2% (w/v) agar-agar (both Merck, Darmstadt, Germany).

### Crossing and Selection of Strains

All crosses were performed under standard conditions as described earlier (Seidl et al, 2009). Progeny (ascospores) from successful crosses were isolated from the lid of the petridish using 10 μl of sterile water. This spore suspension was spread onto 2-3 new plates for isolation of single spore colonies. 10-30 different crosses on individual plates were used for this purpose in each round (depending on efficiency of the previous crosses) and 10-20 colonies were isolated from every single cross yielding several hundred independent progeny from every round of crossing. Single spore colonies were grown on plates and those showing a phenotype resembling QM6a were selected for the next backcrossing step with QM6a. For this step to be successful the strains needed to be of mating type MAT1-2 and be female fertile (because QM6a is female sterile). Since in every round strains with inappropriate phenotypes had to be discarded, this approach yielded an efficiency of successful crosses of less than 20%. Nevertheless, despite the higher number of strains used for crossing, the omission of screening for appropriate mating type (MAT1-1) and fertility would have caused considerably increased effort and time, albeit the result remains the same.

Continuous designation of all strains and their progeny upon crossing in every round enabled follow up individual lines of crosses for the strains analyzed.

After the 10th and final backcrossing step, the resulting strains were tested for mating type by crossing with CBS999.97 MAT1-1 and CBS999.97 MAT1-2 separately and for female fertility crossing with the female sterile strain QM6a was used as control. Fruiting body formation and ascospore discharge of the respective progeny was considered proof of female fertility. In addition, this analysis was repeated with strains evaluated the same way from an additional cross (with female fertile and female sterile strains) in order to confirm fertility or sterility of both strains.

### Sequencing and Identification of SNP Rich Regions

DNA was isolated from three female fertile strains of mating type MAT1-1, which belonged to independent lineages using standard methods. After next generation sequencing of strains, reads of all three strains were mapped in bulk and individually. Mapping was visualized for bulk and individual mappings, for each scaffold individually if necessary using Mapview. Single nucleotide polymorphisms (SNPs) were called with a coverage of 3, a Phred Quality of 20 and Variant frequency was set to 1. SNPs present in all three strains were identified, which indicates that the respective chromosome segments were acquired from CBS999.97 and retained for female fertility. Within the overlapping regions of scaffold 21 enriched in SNPs, recombination blocks are different in all three strains and are not constraint by repeat regions. This suggests that there is a region in the SNP interval that is selected because it aids in fertility. SNP containing genes were annotated for motif conservation and known phenotypes in order to aid in selection of target genes for further analysis.

### Correlation Analysis

Correlation analysis was meant to evaluate the significance of the presence of the QM6a- or CBS999.97 allele of the genes of interest for regaining female fertility by backcrossing of CBS999.97 with QM6a. The backcrossing approach resulted in several female fertile strains with QM6a phenotype and a small portion of genomic content acquired from CBS999.97, which restored female fertility in QM6a. Three of these strains were sequenced (QMF1A, QMF1B and QMF1C).

For evaluation of the significance of selected genes reflecting the three genomic areas we performed another cross between QMF1B and QMF1C with QM6a and isolated 100 random progeny. The resulting strains were tested for mating type and female fertility. 20 female fertile and 20 female sterile strains of mating type MAT1-1 were selected for further analysis, 7 female fertile and 7 female sterile strains of mating type MAT1-2 were used to confirm the results of the analysis of MAT1-1 strains. DNA was isolated from all these strains using standard methods.

We designed primers specific for at least one mutation which enables differentiation between CBS999.97 and QM6a close to the 3′ end of the open reading frame (Tables 1 and 2). Polymerase chain reaction (PCR) amplification was done using GoTaq Polymerase (Promega, Madison, Wis., USA) according to standard protocols.

TABLE 1
Primer sequences used for diagnostic PCR
for evaluation of allele specificity of
genes for female fertility, specific
for the allele present in QM6a.

| | | | SEQ |
|---|---|---|---|
| Gene | Direction | Sequence | ID NO |
| 1 | forward | GGCACAGCTTTCGTGATGAA | 13 |
| | reverse | TGCTATACGGCATCCGAAGG | 14 |
| 2 | forward | CGCAATCGCAATCGCAACAA | 15 |
| | reverse | TATAGCGGGCAATGGTCTCA | 16 |
| 3 | forward | AACTCGATGACGCTGAGCTA | 17 |
| | reverse | AGTTGATGTACCACCCCAGA | 18 |
| 4 | forward | TCAACAGCAGCAGACGAACA | 19 |
| | reverse | CTCTGCTGAAGCTGATGCCG | 20 |
| 5 | forward | TGCAACAGAACCCCCGAGGA | 21 |
| | reverse | CCTTGAGGAAAGTCAGGGGC | 22 |
| 6 | forward | TTGCTCACGACTTGAGCATA | 23 |
| | reverse | TTCTTGGCTCGCCTGTGCGG | 24 |
| 7 | forward | ACCGCACTTCAATCGCTTGG | 25 |
| | reverse | TTCGTTGAGGGGGTGGCGTA | 26 |
| 8 | forward | TTCGTCCATCGACGAGGCTG | 27 |
| | reverse | CAAAGAGTTGTCAACGATGA | 28 |
| 9 | forward | GGGCGCTCAAGCTGTTCCTA | 29 |
| | reverse | TCAAAACGCCCACGGCATCG | 30 |
| 10 | forward | CGATGTCTCGGGCCATGGAA | 31 |
| | reverse | AGCTCCGAAATTTCAAGCAA | 32 |
| 20 | forward | CGCTATACCAAGAGCTGTCATTAATG | 33 |
| | reverse | TCGCTGGGCATGCTGCAGGGAA | 34 |
| 21 | forward | GCACACTCTCGAATCAACAGAAA | 35 |
| | reverse | TGGTAAAGGATTTGTACGGG | 36 |
| 22 | forward | GTTCCGTCACGATGAAGAGG | 37 |
| | reverse | GCTGGGCAGACGGATCTTAA | 38 |
| 23 | forward | ACAGGATGCACTCCAGGTCA | 39 |
| | reverse | TGAGACCGTGCGAGTCGATG | 40 |
| 24 | forward | TTCCTGCGGTGGTGACAACCTCCA | 41 |
| | reverse | TAGACGCGGCCAATCTTCTCGCGA | 42 |
| 25 | forward | AAGTCACCAAATACTTCTCG | 43 |
| | reverse | GTCGGCATCGCACTGCAA | 44 |
| 26 | forward | GATTCGGCGTCTCCATTGCG | 45 |
| | reverse | TGTTGTACATGGCTAGGGAG | 46 |

TABLE 2
Primer sequences used for diagnostic PCR
for evaluation of allele specificity of
genes for female fertility, specific
for the allele present in CBS999.97.

| | | | SEQ |
|---|---|---|---|
| Gene | Direction | Sequence | ID NO |
| 1 | forward | GGCACAGCTTTCGTGATGAA | 47 |
| | reverse | TGCTATACGGCATCCGAAGG | 48 |
| 2 | forward | CGCAATCGCAATCGCAACAA | 49 |
| | reverse | TATAGCGGGCAATGGTCTCA | 50 |
| 3 | forward | AACTCGATGACGCTGAGCTA | 51 |
| | reverse | AGTTGATGTACCACCCCAGA | 52 |
| 4 | forward | TCAACAGCAGCAGACGAACA | 53 |
| | reverse | CTCTGCTGAAGCTGATGCCG | 54 |
| 5 | forward | TGCAACAGAACCCCCGAGGA | 55 |
| | reverse | CCTTGAGGAAAGTCAGGGGC | 56 |
| 6 | forward | TTGCTCACGACTTGAGCATA | 57 |
| | reverse | TTCTTGGCTCGCCTGTGCGG | 58 |
| 7 | forward | ACCGCACTTCAATCGCTTGG | 59 |
| | reverseT | TCGTTGAGGGGGTGGCGTA | 60 |
| 8 | forward | TTCGTCCATCGACGAGGCTG | 61 |
| | reverse | CAAAGAGTTGTCAACGATGA | 62 |
| 9 | forward | GGGCGCTCAAGCTGTTCCTA | 63 |
| | reverse | TCAAAACGCCCACGGCATCG | 64 |
| 10 | forward | CGATGTCTCGGGCCATGGAA | 65 |
| | reverse | AGCTCCGAAATTTCAAGCAA | 66 |
| 20 | forward | CGCTATACCAAGAGCTGTCATTAATG | 67 |
| | reverse | TCGCTGGGCATGCTGCAGGGAA | 68 |
| 21 | forward | GCACACTCTCGAATCAACAGAAAA | 69 |
| | reverse | TGGTAAAGGATTTGTACGGG | 70 |
| 22 | forward | GTTCCGTCACGATGAAGAGG | 71 |
| | reverse | GCTGGGCAGACGGATCTTAA | 72 |
| 23 | forward | ACAGGATGCACTCCAGGTCA | 73 |
| | reverse | TGAGACCGTGCGAGTCGATG | 74 |
| 24 | forward | TTCCTGCGGTGGTGACAACCTCCA | 75 |
| | reverse | TAGACGCGGCCAATCTTCTCGCGA | 76 |
| 25 | forward | AAGTCACCAAATACTTCTCG | 77 |
| | reverse | GTCGGCATCGCACTGCAA | 78 |
| 26 | forward | GATTCGGCGTCTCCATTGCG | 79 |
| | reverse | TGTTGTACATGGCTAGGGAG | 80 |

To test the correlation of the individual genes with fertility, the female fertile wild-type and parental strains CBS999.97 MAT1-1 and CBS999.97 MAT1-2 and the female fertile strains QMF1B and QMF1C (all mating type 1-1) were tested first, as well as two female fertile backcrossed strains (QMF2B and QMF2C) and the female sterile wild-type QM6a for the presence of the alleles of selected genes (17 in total) from all three genomic areas identified to as described above specific for QM6a (sterile) or CBS999.97 (fertile).

Based on these results the specificity of the alleles of the 17 selected genes for female fertility in all the strains of the additional crossing (54 strains of both mating types in total) were evaluated which revealed a characteristic pattern and enabled to determine six genes of one genomic area to be specific for female fertility.

### Determination of Specificity and P-Value

For statistical analysis of PCR analysis of correlation, Fisher's exact test with two tailed p-values (for positive and negative correlation analysis) was applied. (GraphPad Software, Inc., CA, USA:
http://graphpad.com/quickcalcs/contingency1.cfm;
http://www.langsrud.com/stat/fisher.htm; SISA:
http://www.quantitativeskills.com/sisa/statistics/fisher.htm). This test allows even small numbers of experimental data sets and analyzes the significance of the association between two classes. We used the test for analysis of correlation of the presence of the CBS999.97 allele and the feature of female fertility or the QM6a allele and the feature of female sterility, in progeny from crosses of QMF1A, QMF1B and QMF1C with female sterile QM6a.

Finally, all the PCR analysis data were merged and the two tailed p-values for 17 different possible candidate genes were calculated, for which the CBS999.97 alleles are essential for female fertility of QM6a derivatives. Table 3 shows the corresponding results from which it was concluded that the genes FS_4, 5, 6, 7, 8 and 9, corresponding to the six genes mentioned above are specific for female fertility of Trichoderma reesei. The genomic nucleotide sequences of the six genes are provided in SEQ ID NO:1, SEQ ID NO:3, SEQ ID NO:5, SEQ ID NO:7, SEQ ID NO:9 and SEQ ID NO:11.

TABLE 3
Two tailed p-value of the Fisher's exact test
of all strains and all candidate genes.

| Gene | p-value |
|---|---|
| FS_1 | 1.76E−01 |
| FS_2 | 1.72E−01 |
| FS_3 | 7.98E−03 |
| FS_4 | 1.30E−06 |
| FS_5 | 5.03E−09 |
| FS_6 | 2.00E−07 |
| FS_7 | 1.08E−08 |
| FS_8 | 1.59E−09 |
| FS_9 | 3.47E−09 |
| FS_10 | 1.75E−01 |
| FS_20 | 1.76E−01 |
| FS_21 | 5.91E−02 |
| FS_22 | 3.14E−01 |
| FS_23 | 1.00E+00 |
| FS_24 | 3.50E−01 |
| FS_25 | 8.85E−02 |
| FS_26 | 1.76E−01 |

Production of Trichoderma reesei Strains by Transgenic Methods

For fungi, as well as plants, animals, and bacteria, the application of gene transfer technology is quite common and has already resulted in commercial application. Current transformation techniques for fungi have included a combination of CaCl₂ and polyethylene glycol (PEG), electroporation, and particle bombardment to introduce DNA into protoplasts, mycelium, or spores. In recent approaches, several fungi, including filamentous fungi, have been transformed using an Agrobacterium-based transformation system.

### REFERENCE

Agresti A (1992) Modelling patterns of agreement and disagreement. Stat Methods Med Res 1: 201-218
Bowie and Sauer, 1989, Proc. Natl. Acad. Sci. USA 86: 2152-2156
Christensen et al., 1988, Bio/Technology 6: 1419-1422
Cullen et al., 1987, Nucleic Acids Res. 15: 9163-9175
Cunningham and Wells, 1989, Science 244: 1081-1085
Derbyshire et al., 1986, Gene 46: 145; Ner et al., 1988, DNA 7: 127
Debuchy R, Berteaux-Lecellier V, Silar P (2010) Mating systems and sexual morphogenesis in ascomycetes. In Cellular and Molecular Biology of Filamentous Fungi, Borkovich K A, Ebbole D J (eds), 33, pp 501-535. Washington, D.C.: ASM Press
Debuchy R, Turgeon B G (2006) Mating-Type Structure, Evolution and Function in Euascomycetes. In The Mycota I, Kues U, Fischer R (eds), pp 293-323. Berlin: Springer-Verlag
Ford et al., 1991, Protein Expression and Purification 2: 95-107
Gems et al., 1991, Gene 98: 61-67
Hawksworth et al., In, Ainsworth and Bisby's Dictionary of The Fungi, 8th edition, 1995, CAB International, University Press, Cambridge, UK
Hilton et al., 1996, J. Biol. Chem. 271: 4699-4708
Kothe E (1996) Tetrapolar fungal mating types: sexes by the thousands. FEMS Microbiol Rev 18: 65-87
Kuhls K, Lieckfeldt E, Samuels G J, Kovacs W, Meyer W, Petrini O, Gams W, Borner T, Kubicek C P (1996) Molecular evidence that the asexual industrial fungus Trichoderma reesei is a clonal derivative of the ascomycete Hypocrea jecorina. Proc Natl Acad Sci U.S.A. 93: 7755-7760
Lowman et al., 1991, Biochemistry 30: 10832-10837
Malardier et al., 1989, Gene 78: 147-156
Metzenberg R L, Glass N L (1990) Mating type and mating strategies in Neurospora. Bioessays 12: 53-59
Moore-Landecker E (1992) Physiology and biochemistry of ascocarp induction and deveopment. Mycol Res 96: 705-716
Needleman and Wunsch, 1970, J. Mol. Biol. 48: 443-453
Ness et al., 1999, Nature Biotechnology 17: 893-896
Neurath and R. L. Hill, 1979, In, The Proteins, Academic Press, New York
Ni M, Feretzaki M, Sun S, Wang X, Heitman J (2011) Sex in fungi. Annu Rev Genet 45: 405-430
Reidhaar-Olson and Sauer, 1988, Science 241: 53-57
Rice et al., 2000, Trends Genet. 16: 276-277
Sambrook et al., 1989, Molecular Cloning, A Laboratory Manual, 2d edition, Cold Spring Harbor, New York
Schuster A, Schmoll M (2010) Biology and biotechnology of Trichoderma. Appl Microbiol Biotechnol 87: 787-799
Schuster A, Bruno K S, Collett J R, Baker S E, Seiboth B, Kubicek C P and Schmoll M (2012) A versatile toolkit for high throughput functional genomics with Trichoderma reesei. Biotechnology for Biofuels 2012, 5:1
Seidl V, Seibel C, Kubicek C P, Schmoll M (2009) Sexual development in the industrial workhorse Trichoderma reesei. Proc Natl Acad Sci U.S.A. 106: 13909-13914
Taylor J, Jacobson D, Fisher M (1999) THE EVOLUTION OF ASEXUAL FUNGI: Reproduction, Speciation and Classification. Annu Rev Phytopathol 37: 197-246
Tulasne L R, Tulasne C (1865) Selecta fungorum carpologia, Paris: Jussu. Yelton et al., 1984, Proc. Natl. Acad. Sci. U.S.A. 81: 1470-1474,

1. A female fertile variant strain of filamentous fungus derived from a parental female sterile strain comprising at least one of the gene alleles of SEQ ID NO:1, SEQ ID NO:3, SEQ ID NO:5, SEQ ID NO:7, SEQ ID NO:9 or SEQ ID NO:11 or a fragment or a derivative thereof.

2. A female fertile filamentous fungal cell comprising at least one exogenous polynucleotide encoding a mating polypeptide comprising an amino acid sequence at least 60% identical to a sequence chosen from the group of sequences consisting of SEQ ID NO:2, SEQ ID NO:4, SEQ ID NO:6, SEQ ID NO:8, SEQ ID NO:10 and SEQ ID NO:12; preferably the amino acid sequence is at least 65% identical, more preferably it is at least 65%, 70%, 75%, 80%, 85%, 90%, 95%, 97%, 98%, 99% or 100% identical to SEQ ID NO:2, SEQ ID NO:4, SEQ ID NO:6, SEQ ID NO:8, SEQ ID NO:10 or SEQ ID NO:12.

3. A female fertile filamentous fungal cell comprising at least one exogenous polynucleotide, wherein the cDNA of which having a nucleotide sequence at least 60% identical to the joined coding region(s) in SEQ ID NO:1, SEQ ID NO:3, SEQ ID NO:5, SEQ ID NO:7, SEQ ID NO:9 or SEQ ID NO:11; preferably the nucleotide sequence is at least 65%, 70%, 75%, 80%, 85%, 90%, 95%, 97%, 98%, 99% or 100% identical to the joined coding region(s) in SEQ ID NO:1, SEQ ID NO:3, SEQ ID NO:5, SEQ ID NO:7, SEQ ID NO:9 or SEQ ID NO:11.

4. A female fertile filamentous fungal cell comprising at least one exogenous polynucleotide having a nucleotide sequence at least 60% identical to SEQ ID NO:1, SEQ ID NO:3, SEQ ID NO:5, SEQ ID NO:7, SEQ ID NO:9 or SEQ ID NO:11; preferably the nucleotide sequence is at least 65%, 70%, 75%, 80%, 85%, 90%, 95%, 97%, 98%, 99% or 100% identical to SEQ ID NO:1, SEQ ID NO:3, SEQ ID NO:5, SEQ ID NO:7, SEQ ID NO:9 or SEQ ID NO:11.

5. A female sterile filamentous fungal cell wherein at least one exogenous polynucleotide encoding a mating polypeptide has been inactivated, said mating polypeptide comprising an amino acid sequence at least 60% identical to a sequence chosen from the group of sequences consisting of SEQ ID NO:2, SEQ ID NO:4, SEQ ID NO:6, SEQ ID NO:8, SEQ ID NO:10 and SEQ ID NO:12; preferably the amino acid sequence is at least 65% identical, more preferably it is at least 65%, 70%, 75%, 80%, 85%, 90%, 95%, 97%, 98%, 99% or 100% identical to SEQ ID NO:2, SEQ ID NO:4, SEQ ID NO:6, SEQ ID NO:8, SEQ ID NO:10 or SEQ ID NO:12.

6. A female sterile filamentous fungal cell, wherein at least one polynucleotide encoding a mating polypeptide has been inactivated and said at least one polynucleotide has a nucleotide sequence at least 60% identical to the joined coding region(s) in SEQ ID NO:1, SEQ ID NO:3, SEQ ID NO:5, SEQ ID NO:7, SEQ ID NO:9 or SEQ ID NO:11; preferably the nucleotide sequence has a nucleotide sequence at least 65%, 70%, 75%, 80%, 85%, 90%, 95%, 97%, 98%, 99% or 100% identical to the joined coding region(s) in SEQ ID NO:1, SEQ ID NO:3, SEQ ID NO:5, SEQ ID NO:7, SEQ ID NO:9 or SEQ ID NO:11.

7. A female sterile filamentous fungal cell, wherein at least one polynucleotide encoding a mating polypeptide has been inactivated and said at least one polynucleotide has a nucleotide sequence at least 60% identical to SEQ ID NO:1, SEQ ID NO:3, SEQ ID NO:5, SEQ ID NO:7, SEQ ID NO:9 or SEQ ID NO:11; preferably the nucleotide sequence is at least 65%, 70%, 75%, 80%, 85%, 90%, 95%, 97%, 98%, 99% or 100% identical to SEQ ID NO:1, SEQ ID NO:3, SEQ ID NO:5, SEQ ID NO:7, SEQ ID NO:9 or SEQ ID NO:11.

8. The filamentous fungal cell of any preceding claim which is an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticiffium, Volvariella, or Xylaria cell; preferably an Aspergillus or Trichoderma cell.

9. The filamentous fungal cell according to claim 8 which is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zona turn, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticula turn, Fusarium roseum, Fusarium sambucinum, Fusarium sarcochroum, Fusarium sporotrichioides, Fusarium sulphureum, Fusarium torulosum, Fusarium trichothecioides, Fusarium venenaturn, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianurn, Trichoderma koningii, Trichoderma longibrachiatum, Trichoderma reesei, or Trichoderma viride cell; preferably an Aspergillus niger, Aspergillus oryzae or Trichoderma reesei cell.

10. The filamentous fungal cell of any preceding claim, comprising a polynucleotide encoding a polypeptide of interest, preferably the polypeptide of interest is a hormone, enzyme, receptor or portion thereof, antibody or portion thereof, or reporter; more preferably the polypeptide of interest is a hydrolase, isomerase, ligase, lyase, oxidoreductase, or transferase enzyme; most preferably the polypeptide of interest is an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

11. The filamentous fungal cell of claim 10, wherein the polynucleotide encoding the polypeptide of interest is exogenous or endogenous to the cell.

12. The filamentous fungal cell of claim 10 or 11, wherein the polynucleotide encoding the polypeptide of interest is present in the cell in two or more copies; preferably integrated into the chromosome of the cell.

13. A method for converting a female sterile filamentous fungal cell to a female fertile cell, said method comprising a step of transforming the sterile cell with at least one polynucleotide encoding a mating polypeptide comprising an amino acid sequence at least 60% identical to a sequence chosen from the group of sequences consisting of SEQ ID NO:2, SEQ ID NO:4, SEQ ID NO:6, SEQ ID NO:8, SEQ ID NO:10 and SEQ ID NO:12, whereby the cell becomes fertile; preferably the amino acid sequence is at least 65% identical, more preferably it is at least 65%, 70%, 75%, 80%, 85%, 90%, 95%, 97%, 98%, 99% or 100% identical to SEQ ID NO:2, SEQ ID NO:4, SEQ ID NO:6, SEQ ID NO:8, SEQ ID NO:10 or SEQ ID NO:12.

14. A method for converting a female sterile filamentous fungal cell to a female fertile cell, said method comprising a step of transforming the sterile cell with at least one polynucleotide encoding a mating polypeptide, wherein said at least one polynucleotide has a cDNA nucleotide sequence at least 60% identical to the joined coding region(s) in SEQ ID NO:1, SEQ ID NO:3, SEQ ID NO:5, SEQ ID NO:7, SEQ ID NO:9 or SEQ ID NO:11; preferably the nucleotide sequence is at least 65%, 70%, 75%, 80%, 85%, 90%, 95%, 97%, 98%, 99% or 100% identical to the joined coding region(s) in SEQ ID NO:1, SEQ ID NO:3, SEQ ID NO:5, SEQ ID NO:7, SEQ ID NO:9 or SEQ ID NO:11.

15. A method for converting a female sterile filamentous fungal cell to a female fertile cell, said method comprising a step of transforming the sterile cell with at least one polynucleotide encoding a mating polypeptide, wherein said at least one polynucleotide has a nucleotide sequence at least 60% identical to SEQ ID NO:1, SEQ ID NO:3, SEQ ID NO:5, SEQ ID NO:7, SEQ ID NO:9 or SEQ ID NO:11; preferably the nucleotide sequence is at least 65%, 70%, 75%, 80%, 85%, 90%, 95%, 97%, 98%, 99% or 100% identical to SEQ ID NO:1, SEQ ID NO:3, SEQ ID NO:5, SEQ ID NO:7, SEQ ID NO:9 or SEQ ID NO:11.

16. A method for converting a female fertile filamentous fungal cell to a female sterile cell, said method comprising a step of inactivating at least one polynucleotide encoding a mating polypeptide, wherein said mating polypeptide comprises an amino acid sequence at least 60% identical to a sequence chosen from the group of sequences consisting of SEQ ID NO:2, SEQ ID NO:4, SEQ ID NO:6, SEQ ID NO:8, SEQ ID NO:10 and SEQ ID NO:12, whereby the cell becomes fertile; preferably the amino acid sequence is at least 65% identical, more preferably it is at least 65%, 70%, 75%, 80%, 85%, 90%, 95%, 97%, 98%, 99% or 100% identical to SEQ ID NO:2, SEQ ID NO:4, SEQ ID NO:6, SEQ ID NO:8, SEQ ID NO:10 or SEQ ID NO:12.

17. A method for converting a female fertile filamentous fungal cell to a female sterile cell, said method comprising a step of inactivating at least one polynucleotide encoding a mating polypeptide, wherein said polynucleotide has a cDNA nucleotide sequence at least 60% identical to the joined coding region(s) in SEQ ID NO:1, SEQ ID NO:3, SEQ ID NO:5, SEQ ID NO:7, SEQ ID NO:9 or SEQ ID NO:11; preferably the nucleotide sequence is at least 65%, 70%, 75%, 80%, 85%, 90%, 95%, 97%, 98%, 99% or 100% identical to the joined coding region(s) in SEQ ID NO:1, SEQ ID NO:3, SEQ ID NO:5, SEQ ID NO:7, SEQ ID NO:9 or SEQ ID NO:11.

18. A method for converting a female fertile filamentous fungal cell to a female sterile cell, said method comprising a step of inactivating at least one polynucleotide encoding a mating polypeptide, wherein said polynucleotide has a nucleotide sequence at least 60% identical to SEQ ID NO:1, SEQ ID NO:3, SEQ ID NO:5, SEQ ID NO:7, SEQ ID NO:9 or SEQ ID NO:11; preferably the nucleotide sequence is at least 65%, 70%, 75%, 80%, 85%, 90%, 95%, 97%, 98%, 99% or 100% identical to SEQ ID NO:1, SEQ ID NO:3, SEQ ID NO:5, SEQ ID NO:7, SEQ ID NO:9 or SEQ ID NO:11.

19. The method of any of claims 13 to 18, wherein the host cell comprises a polynucleotide encoding a polypeptide of interest; preferably the polypeptide of interest is a hormone, enzyme, receptor or portion thereof, antibody or portion thereof, or reporter; more preferably the polypeptide of interest is a hydrolase, isomerase, ligase, lyase, oxidoreductase, or transferase enzyme; most preferably the polypeptide of interest is an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

20. The method of claim 19, wherein the polynucleotide encoding the polypeptide of interest is exogenous or endogenous to the cell.

21. The method of claim 19 or 20, wherein the polynucleotide encoding the polypeptide of interest is present in the cell in two or more copies; preferably integrated into the chromosome of the cell.

22. A method of producing a polypeptide of interest, said method comprising cultivating a filamentous fungal host cell according to any one of claims 1 to 12 under conditions conducive for the expression of the polypeptide; and, optionally recovering the polypeptide.

23. The variant strain of claim 1, wherein the filamentous fungus is a filamentous heterothallic ascomycete, preferably selected from Trichoderma sp., Aspergillus sp. or Neurospora sp.

24. The variant strain of claim 1, wherein the filamentous fungus is Trichoderma reesei.

25. The variant of claim 24, wherein the Trichoderma reesei is QM6a.

26. An isolated nucleic acid according to SEQ ID NO:1, SEQ ID NO:3, SEQ ID NO:5, SEQ ID NO:7, SEQ ID NO:9 or SEQ ID NO:11.

27. An expression system containing at least one gene according to SEQ ID NO:1, SEQ ID NO:3, SEQ ID NO:5, SEQ ID NO:7, SEQ ID NO:9 or SEQ ID NO:11, or up to 2, 3, 4, 5 or 6 genes according to SEQ ID NO: 1, SEQ ID NO:3, SEQ ID NO:5, SEQ ID NO:7, SEQ ID NO:9 or SEQ ID NO:11, or mixtures thereof.

28. A variant strain according to any one of claims 1 to 12, which is obtainable by a process comprising the steps of
introducing into a cell of a parental fungus at least one gene of SEQ ID NO:1, SEQ ID NO:3, SEQ ID NO:5, SEQ ID NO:7, SEQ ID NO:9 or SEQ ID NO:11 and expressing said at least one gene in said cell.

29. The process according to claim 28, wherein the filamentous fungus is Trichoderma reesei, specifically QM6a.

30. The process according to claim 28 or 29, wherein the filamentous fungus comprises a gene encoding a heterologous protein.

31. A female fertile variant strain obtained by a process of anyone of claims 28 to 30.

32. A method of producing a heterologous protein comprising the steps of cultivating the female fertile variant strain of any one of claims 1 to 12 and, optionally, recovering the heterologous protein.

33. The heterologous protein according to claim 32 for use in industrial or pharmaceutical applications, preferably in food industry.

34. The filamentous fungus Trichoderma reesei deposited on Jul. 23, 2012 under Accession No. DSM 26183, DSM 26184, DSM 26185 or DSM 26186.


SEQUENCE LISTING (80 sequences)

SEQ ID NO: 1 - DNA, 1902 bp, Trichoderma reesei
  CDS (1)..(83)
  Intron (84)..(161)
  CDS (162)..(1899): CBS_FS4_ORF encodes putative mating polypeptide
atgtcggaccagggcaactacaacagctggaccaaggccggactcatccagcgggtcaaggagctggagaagcagctcaaggcgtcgtcatcatttcacgagcccgcttcaacagcagcagacgaacagcaacagcaacagccacagccgccatcacagagcgaacaacatggacaggagcagaagcaggacgaaaacgaacacgacgaggcctccgccgccgcccaaccggccaagaagcccaaatccaagggcaagaagaagatggacccctcaaagtacgccacgcggtacattgcgctgaagctggcgtacctgggcaagaactttggcggcttcgagttccaggccatgggcaaccagccgtccatcgaggaggagctgtggaacgcgctgaccaaggcgtgcctcatcttccccgaggacgagcggatcgtcgactttgagtgctgcgagtattccaagtgcgggcggacggaccgcggcgtgagcgcctttgggcaggtcattgggctgagggtgcggagcaacaggccgttgcccaagaagccggccgagggcgccgaggagatggatgtggaacagacagggcccccggcatcagcttcagcagagcaggagacgcagccgcaacaagacaaagacgacgacaaccaggccaaggcaaaggccaaggcaaagaagcagccggagcccaccaggccctttgacgacatccgcgacgagatcccctacccgcacgtcctcaaccggctgctccccaaggagatccggatcctcgcctggtgcccgtcgccgccgcccggcttctccgcccgcttctcgtgccgggagcggcagtaccggtatttcttcacgcagccggcctttgcccccgcgccgtctcacgtcgacaacacagcagcagcagcagcagcagcagcaaccgggcaaggaggagccaacggcaaggctgtcaagtccggttggctggacattgacgccatgcgcgacgcggcgaagcggtacgagggcgagcacgacttccgcaacctgtgcaagattgacccggccaagcaaatcaccaacttcaagcggcgcatgttcgagtcggacattgtcgaggtcgaggacgccgcgtcggcgctgccgtacctccaagcggcgggctttgcccccgagaaccttgaggcgctggactcttcgtcgtcgtcgtcgtcgtcgtctgcttcgggggccagaaagggggtgtatcccaaggtgtactatttccacgtccgcgggtccgcgttcctgtggcatcagatccggcacatggttgccgtgctgttcctcgtgggccaggggctggagaagccgtcggtggtgagcgagctgctcgacgtggacaagaacccgcggcggccaaactacgtcatggcggacgaggtgccgctggtgctctgggactgcatcttcccgtctgatctgagcgcgtcgacggcggcccatcgcgaggacgacgcgctgcggtgggtgtacgtcggggatgacgggccgagcgggcggttcggccagtttggcatcatggacgacgcgtggcagatgtggcgcgagaagaagatggacgaggtgcttgccgggcagctgctgcagcatatctcgaagcagggcaataacactgctacttcgtccaaggcggcgacgacgacgacgacgacgacgaccgcggcggcgagcgtaaaggtgtttgagggcggtaacggcgggaggctgagcggcaagtatcctggcatcatgaacatgaagttgctggagagcgcggacgagcagaacgacaagtatgccaagagaaaggggtatgcggatgcggaggatatgagggcgaagaaggggttcaagagtgccaaggcggaggaggatatcaccatggcggatgaatga

SEQ ID NO: 2 - protein, 607 aa, Trichoderma reesei
MSDQGNYNSWTKAGLIQRVKELEKQLKAEQHGQEQKQDENEHDEASAAAQPAKKPKSKGKKKMDPSKYATRYIALKLAYLGKNFGGFEFQAMGNQPSIEEELWNALTKACLIFPEDERIVDFECCEYSKCGRTDRGVSAFGQVIGLRVRSNRPLPKKPAEGAEEMDVEQTGPPASASAEQETQPQQDKDDDNQAKAKAKAKKQPEPTRPFDDIRDEIPYPHVLNRLLPKEIRILAWCPSPPPGFSARFSCRERQYRYFFTQPAFAPAPSHVDNTAAAAAAAATGQGGANGKAVKSGWLDIDAMRDAAKRYEGEHDFRNLCKIDPAKQITNFKRRMFESDIVEVEDAASALPYLQAAGFAPENLEALDSSSSSSSSSASGARKGVYPKVYYFHVRGSAFLWHQIRHMVAVLFLVGQGLEKPSVVSELLDVDKNPRRPNYVMADEVPLVLWDCIFPSDLSASTAAHREDDALRWVYVGDDGPSGRFGQFGIMDDAWQMWREKKMDEVLAGQLLQHISKQGNNTATSSKAATTTTTTTTAAASVKVFEGGNGGRLSGKYPGIMNMKLLESADEQNDKYAKRKGYADAEDMRAKKGFKSAKAEEDITMADE

SEQ ID NO: 3 - DNA, 5121 bp, Trichoderma reesei
  CDS (1)..(84): CBS_FS5_ORF encodes putative mating polypeptide
atgccgtccgccgcggacctcaaatacttcatcccgtctgctgccacggcgtccatcttcctctatgcgcagggaccaacgatagtctgttgccatcacgacacgctcaccatcgaccggaggttctcacgccatgtcgacgatgtccagctgcttgccgtggataaccagagcgacatgggcgctggccggttcgtcgtgagctatgacgctggccagactgccattgtgtgggatctaatgacgggcgatgagatttcgcgtttcgtttcgtacgagacgctcacagtcgccgcttggatgcggaatggaaacgttgctttcggtgagcggaaccagagatcgccctccccctctccccttaagctggtgacatgaggtggctaacggattgggcttccaggcaacacacagggaaacatcatcatgtttgaacctactacatccgagcatatctcggctcggacgctagaccagattgcagtgacagcattagccccttcgtctgactgccggacctttgcgatcgggtaagttggtgttcgaccatgaatcgagaagtctgtcgcgaatgtgccacggcgtactgattccatccgatgtagctaccagaacggatctctcttggtcgcaactctgcagcctcggttcaccatcttacacaacctgacgacctcgagagggccatcgcccattgtcaccctcgcctggcacgcgtcctcctcgaggcagaaatcagacatgctggccgtacagacgcacgatggcgacctgcgggtctggagtgtggccaagtcgtacagcgccgaagatcccgccaaggtcgtcagggtgctccgcaggaacgagaacttcctggctgggcccaattggatggggtggtccaagaacggccgcatcatccaatactcggactcgtaagtcccctatcaactggacacagcctaagcgcgcggcctcccgcttctgctggtttggttttttgttgtgctgacctcgttgtgcagggaaacgttttcgtgggacgtgagaaccaaacatgtcacaaaggactccatcccgacccttgagcatgttaaaggcctggccgtctatggcccaggagccagtctcttcacccttggaccgaacaacaccgtgcagcagtttgacctcaactccccagccatcatggtggcaaacgtgcagcaccccgcgggcgtccttccgccatctccacccacctccgaggagacagggggcaggtcggtgcactctgccacgaccattcatacctcggaatccgagtcgagctctgtccccctcgagatgggcatctctgaaagcgatgacgaccacctatcccctttccagcggctagcaaagcgcaatgcccccgaagccaggaacgaggtgtacgatacaggcagcgcggcctctagccagagcggcgtgtcatccttgtcaaagtcttcggccagctctcgtacgcctggcaggcaggccagctcgctcaggtcgcggggaatgacggaggggacatacatctccgctggttcatccatgaaaacttcgacagttggccagcgcgaggcggacaactactccatgggatacacactccctagcaccagcggtccatcattggcgtcgtcccggtccaggcatcggccctctcgtctcagacacgaagtaccgaggagtcctgacgaggccaacgtgcaggacctgttcaaatacactcgctcgcggcttagcgacctcccgtacaagcacccgatgccaacgcaacgatctcatcctaccaatgacgatttgcgccgacagatgctcagcactatctttggctggaacaaggaagtagaagacctcattcgagacgagatgagtcgctatcccgcagggtcggcgaaccgaatcttgctagccaagtggctgggcgacattgacgcagacatcatggccgcgggttcccagaacatgacttcccaagactggatgctgctggcactgagcggcattggcggccaggcatcccagcacaagctcggccgagtctatgtccaacgcttgctagagaatggcgatgtccatgttgcggtgacgattatgcttgggatgggcgactataacgatgccattgaggtctacatctcgcacaagcggtatatggaggccctcattctcacctgcgtggcttttcccagcgtctgggagcgtcaagctgccattgtgcgcaaatggggtgagtgggcggtcaagcatggccagcagcaactggcaattcgttgctttgcctgtaccgaccaggagtcctcggagccttggacctctccatcggctgcccagctaaacttccaaaacatcaccccgagcatccccgaggtgctgagccctccgctgtcgcccccgggcattcagagaggcccccagcgcagcgtcgccaaggcatcggcactgaagctgattacgtcctttggagatcccacccaaaaggccaagttctactcgcaggctgacggcggccagacaccaattgcagctggagtgacgcccattgccgagtcggccatctctccgggaggcgcctatgatcccgcaactgcgttcctccggccgtcgggcaatagcaggttcaacacgccaacatcagcccgccctatcggtcagggcttcagccgcggacggctgccctccatcggagaagccaacaagcccctggataacattgcaagcattgtggacgcaccccagaaacggccgagccattctcgcaagtcgtccgcccctcaagataacatggcaaccgggctcgccatgcagcgcgctgctacggccagcccaatgatgatgagggaccagtaccagcgcgccgtgcaagggtacggaggagagcgaccgccgtctcccgaccataacatcatgagcagactgcaagaggtccattcggcacagcgaaacggctcccgtgaccggattcctgctcaccttagcctgcagctgcagacgatgcagccgccatcgatggaggcaatgtctcctgagcaatccggcgcctcatccgctcgcttccactggccgtctcgtcgccgcggtactggtcctagttccgcatctgtggccggttccatgacatccacctccagcgctggccggagtcacaaatcgaacgctcgacaaagggatgattacatccatagcctggaagcggctcagcactattccaggagagctggaagccggaccggaagcaaggaacggacacgagacgcgtccaccggccgccacgccagccgagagcgacgaaccaagtcgcgcgacccttcggaagagcgcggcagggcttcagccagatcctggacaaggccaaagcgatcacccacatccccagttccaatgtcccccgaagacttggccatgctgagcaaccggacattcgacaactccgtggaacccctcacgattcgaaaggcgagcgtggcgaaaggcaaggccagcgggcggacatccagccgcggaaggggtggatcagtaccgcggtcaccgccgtccccggtgccgctgtcggcaacagccctgcattatcagggatcggaggacgaggaggactttagagcagccatgcgggcgcaggaagagttcagggccaagcacagccgcagcgtcggccacaacgtcaattctcccgccgtaagccggcgtgagcattcggaaagccgacgaaaggaagcgactgagactcgagaggctgcacccgtggtcttgagccagacagtatatggacgagccgcttccaccgagcacgccggcgatctgaagaagatgaaggacgagagacagcggaagaaggaacaggctgcgcgagagctagaagagcgtaggaagtctctggccaagcgacagctcggtccccagattccccatccaagccagatctctcccgggagaccgccggttctggtagaggccgacgatgaaaagctgccggatgatctgccgccgcgctctgcaacagaacccccgaggacggcggagccgccgaggagcatgtatgctcaaaacaggccgcagattggcctgcctgccactcccaaggcgatgaggctcattatccggtctgacgagaatcagtacgaggacctccctgctccgccggtgccggcgacgttttctcaaaagtattcgccgcagaactcaccccagtactctccgaaatacacgttgggcagtagtgtttatggagaggagcagaagcagcagcagcagcagcagcaacagcagggacagcagcagcagcaacaacgacagcaacaacaacagcagcagcagcaacagcaacagcagcagcagcaacaacaacaactctttcatcagaaagaggaggagccgccgttgacactgctgccatcgactgtctatcagctgccgtccactgtctaccaaccgccgtctcgccccatgattccgcgaagcatgtcggcaccgatccctgatgagccacctcacgcaattcgatacgggagaaagtcgagtgctaacgagggcagaggcctcgacgatattgtagagacggagtggcgtcgacaagtgaacaacctggtgccgcctccaccaccacctcccccttcggcgcccctgactttcctcaaggagctgcagcatctagccgtgccgccccctcctccgccagcaccgctgccacacgtgcgacgccagccgcccgtggctggaacactggcctcgggcatgatcgaaattgtcatggacgatgacgaggcagaagagcccatgtcggctgctcccaacgacggcatggtgccggtgctcgcccagcccgaaccccctgtcaagggccacaaccgcggtcgcagcctcggggagggcagcctttccgggcgtaggaccaaggcgacggagcgcctccgctcgggcagccgcagccgcaaaggatccatcggcgtcatgtcgccccctctcgagatgtacggcggtgagggcaatgccaactcgctgaaccagctcaggtcgcccgtggtgggcaatcctccgcctgtgctgtatgaccgggacgccattcgatcgcccattgaggggcatggccgcaagatgtcgatggggctgcacgagaacggcatgttttag

SEQ ID NO: 4 - protein, 1503 aa, Trichoderma reesei
MPSAADLKYFIPSAATASIFLYAQGPTITAIVWDLMTGDEISRFVSYETLTVAAWMRNGNVAFGNTQGNIIMFEPTTSEHISARTLDQIAVTALAPSSDCRTFAIGPSPIVTLAWHASSSRQKSDMLAVQTHDGDLRVWSVAKSYSAEDPAKVVRVLRRNENFLAGPNWMGWSKNGRIIQYSDSETFSWDVRTKHVTKDSIPTLEHVKGLAVYGPGASLFTLGPNNTVQQFDLNSPAIMVANVQHPAGVLPPSPPTSEETGGRSVHSATTIHTSESESSSVPLEMGISESDDDHLSPFQRLAKRNAPEARNEVYDTGSAASSQSGVSSLSKSSASSRTPGRQASSLRSRGMTEGTYISAGSSMKTSTVGQREADNYSMGYTLPSTSGPSLASSRSRHRPSRLRHEVPRSPDEANVQDLFKYTRSRLSDLPYKHPMPTQRSHPTNDDLRRQMLSTIFGWNKEVEDLIRDEMSRYPAGSANRILLAKWLGDIDADIMAAGSQNMTSQDWMLLALSGIGGQASQHKLGRVYVQRLLENGDVHVAVTIMLGMGDYNDAIEVYISHKRYMEALILTCVAFPSVWERQAAIVRKWGEWAVKHGQQQLAIRCFACTDQESSEPWTSPSAAQLNFQNITPSIPEVLSPPLSPPGIQRGPQRSVAKASALKLITSFGDPTQKAKFYSQADGGQTPIAAGVTPIAESAISPGGAYDPATAFLRPSGNSRFNTPTSARPIGQGFSRGRLPSIGEANKPLDNIASIVDAPQKRPSHSRKSSAPQDNMATGLAMQRAATASPMMMRDQYQRAVQGYGGERPPSPDHNIMSRLQEVHSAQRNGSRDRIPAHLSLQLQTMQPPSMEAMSPEQSGASSARFHWPSRRRGTGPSSASVAGSMTSTSSAGRSHKSNARQRDDYIHSLEAAQHYSRRAGSRTGSKERTRDASTGRHASRERRTKSRDPSEERGRASARSWTRPKRSPTSPVPMSPEDLAMLSNRTFDNSVEPLTIRKASVAKGKASGRTSSRGRGGSVPRSPPSPVPLSATALHYQGSEDEEDFRAAMRAQEEFRAKHSRSVGHNVNSPAVSRREHSESRRKEATETREAAPVVLSQTVYGRAASTEHAGDLKKMKDERQRKKEQAARELEERRKSLAKRQLGPQIPHPSQISPGRPPVLVEADDEKLPDDLPPRSATEPPRTAEPPRSMYAQNRPQIGLPATPKAMRLIIRSDENQYEDLPAPPVPATFSQKYSPQNSPQYSPKYTLGSSPPLTLLPSTVYQLPSTVYQPPSRPMIPRSMSAPIPDEPPHAIRYGRKSSANEGRGLDDIVETEWRRQVNNLVPPPPPPPPSAPLTFLKELQHLAVPPPPPPAPLPHVRRQPPVAGTLASGMIEIVMDDDEAEEPMSAAPNDGMVPVLAQPEPPVKGHNRGRSLGEGSLSGRRTKATERLRSGSRSRKGSIGVMSPPLEMYGGEGNANSLNQLRSPVVGNPPPVLYDRDAIRSPIEGHGRKMSMGLHENGMF

SEQ ID NO: 5 - DNA, 1202 bp, Trichoderma reesei
  CDS (1)..(632): CBS_FS6_ORF encodes putative mating polypeptide
atgccgccgaagaaggagaagcctgccgccggcaagaagccgtctgccgccaagctggtggaggaccggacgtttggcatgaagaacaagaaaggagcgcaggcgcagagacagattcagcaaatgacggccaacttgaagggcagtggcagcgccgaagacagacggaaggcggccgaaaaggcacagcgtgagaaggagaagaaggcggcagaggaggccaggagagagaccgaggctctcttgaacaagcccgcccagatccaaaaggtgccctttggcgtggaccccaagacggtcgtctgcatcttctacaagaagggcgactgcgagaagggcaagaagtgcaagtttgctcacgacttgagcatagagagaaagacggagaagaagaacttatacacggatgtcaggcaggaggaggaggagaagaagaagcaggagacgtctgccgactggacagaggagcagctgctcaaggttgtgctgtcaaagaagggtaaccagaagacaacgacagacaaagtctgcaagtacttcatccaagccatcgaggatggaaagtacggttggttctggatctgtccaaatggtggtgataagtgcatgtacaagcatgctcttccgcccgggtacgtcaatctaaccttggtctatacatggtattggttactgacgtcaactattacaggttcgtgctcaagacgaaagaacagagagcagcggagaaggcgctgcttgataagtcaccgctcaagactctgactcttgaggatttcctggaatcggaacgccacaagctcacaggaaaactgacgccagtcactcccgagacgtttgccaagtggaagaaggaacgcttggataagaaggctgctgaggaacaggcccgcaaggccaaggacaacactggccgtgctctgttcgaaagcggcaagtggaaggacgttgattccgaggcagaaagtgaagacgaagaggatgatacctggaacctcgagaagcttcgtcgcgagactgaagctctccggcagaagaaggaagaagagcgactggcaacgctgcacggtggacaggtgcccatcagtacacctgatacgccggaagaagcagctgggagcgatgagggcaacggcgatgctagcgggactgcggaagaagcatcagccgcacaggcgagccaagaagtaccggcctcttga

SEQ ID NO: 6 - protein, 380 aa, Trichoderma reesei
MPPKKEKPAAGKKPSAAKLVEDRTFGMKNKKGAQAQRQIQQMTANLKGSGSAEDRRKAAEKAQREKEKKAAEEARRETEALLNKPAQIQKVPFGVDPKTVVCIFYKKGDCEKGKKCKFAHDLSIERKTEKKNLYTDVRQEEEEKKKQETSADWTEEQLLKVVLSKKGNQKTTTDKVCKYFIQAIEDGKYGWFWICPNGGDKCMYKHALPPGFVLKTKEQRAAEKALLDKSPLKTLTLEDFLESERHKLTGKLTPVTPETFAKWKKERLDKKAAEEQARKAKDNTGRALFESGKWKDVDSEAESEDEEDDTWNLEKLRRETEALRQKKEEERLATLHGGQVPISTPDTPEEAAGSDEGNGDASGTAEEASAAQASQEVPAS

SEQ ID NO: 7 - DNA, 2121 bp, Trichoderma reesei
  CDS (1)..(13): CBS_FS7_ORF encodes putative mating polypeptide
atggtccaggtcggtacgttgttcgaaggccttccgtcgcgtcggatcatgcgccctggagagcgagcaatgggctcccgttactgaccgcacttcaatcgcttggcgtgttagaccggtccgagtatgcttcttcgcctgctccgttcctttccaaacacctcgcgctgtctgctaacgccactccgtctcacagatcagcctcgccgttctggaggccgccgccacgagacggcaaccctagaagaagcgttacgaacccggcaggtcgagatggctcgaatctcgtcgttggcgcggcgtattctcatgcggacatgctcaagttcgattccttgtatgagacccgtatatcggcacgctgtccccgtcccgccatggctgaccttgagcccaggaacctctccagctcttcttcgcctcgtccccggacccccccttcgacatcgtcctcgagacatcgcatgcccgaaactacgccaccccctcaacgaagcgcactggcttcctcctccccaccgttcaagagctacatcaacttcctgtcgaataccaatgatgattggaaggccgatgaagatgagatgctgggatacgaagatgacgacggtgatgattttgggctccccagtctctcaaacatgaagcgaagatcgaagcgaatagctacccaaaacaacaggtcggagccatggcaggaagcgttgtcccccgtgggcgacggttcttttggccgagtccaagcacggcgttattccaacagcgccgatattgcagctgagcgtccggcatcaacatatccaatgcccaagaaaagcgagggcaagattctccgaccacagtataaagatatcctgaagggtaagaatggcggctcctggcttgtcgtctgcttacacggagtactgaccctttgactagatcccgcaaatgcgctccatctgatcagctatccctctatacccagcagtgcgccgcagaaagaagccgacgcgatcaactcgcgaataacgcgcataaacaagttcaaaaggctgctacaggcgtcttcaatatcgcttcctgaccttcgctcgctagcatggtcgggtgtaccgcacgaagtccgcgcaatgacctggcagctcctccttagctatctccctacgaatagtgagcggcgcgttgccactctggagaggaagcggaaggagtatctggacggcgttaagcaagcgttcgaaagaggggggacagcagccagcagctcttccgccggaaaagccaggggcttggatgaagctgtctggcatcagatcagcattgacatacccaggacgaatccccacattgagctgtatagctatgaggcgacacagaggtcgctggaacgcatcctctatgtttgggccgtcaggcatcccgccagcggatacgtgcagggcatcaacgatctggtcacgccattctgggaagtgttcttggggctctacattaccgatccagatatcgagacgggcatggatcctgggcaattgcccaagtccgtcctggatgctgtggaagcagactcgttctggtgcctcagcaagcttctcgatggcattcaagatcactatattgttgctcaaccgggtattcaaaggcaggtggcagccctgcgggatctcactgccaggattgactccaacttgtcaaagcatcttgagcaagaaggcgtcgagttcatacagtttagtttccggtggatgaactgtcttcttatgcgcgaaatcagcgtcaagaacacaatccgtatgtgggatacataccttgtaagtgaaatgcctttgctttcttaaacccccccacagtccacggcccagctaacgacgatgggaccaggccgaagaacagggcttttcagaattccatttatatgtttgcgctgcgctcctcgtaaagtggtctgacaaactggtcaagatggactttcaggaggtcatgatgttcctacagagcctcccgacgaagacatggacggagaaggacattgagcttctcctcagcgaggctttcatctggcagagcctctacaaaggctcagctgctcacctgaagggccagccgcagcgtcccttgcttgggaacctccagttatag

SEQ ID NO: 8 - protein, 585 aa, Trichoderma reesei
MVQVDRSESASPFWRPPPRDGNPRRSVTNPAGRDGSNLVVGAAYSHADMLKFDSLNLSSSSSPRPRTPPSTSSSRHRMPETTPPPQRSALASSSPPFKSYINFLSNTNDDWKADEDEMLGYEDDDGDDFGLPSLSNMKRRSKRIATQNNRSEPWQEALSPVGDGSFGRVQARRYSNSADIAAERPASTYPMPKKSEGKILRPQYKDILKDPANALHLISYPSIPSSAPQKEADAINSRITRINKFKRLLQASSISLPDLRSLAWSGVPHEVRAMTWQLLLSYLPTNSERRVATLERKRKEYLDGVKQAFERGGTAASSSSAGKARGLDEAVWHQISIDIPRTNPHIELYSYEATQRSLERILYVWAVRHPASGYVQGINDLVTPFWEVFLGLYITDPDIETGMDPGQLPKSVLDAVEADSFWCLSKLLDGIQDHYIVAQPGIQRQVAALRDLTARIDSNLSKHLEQEGVEFIQFSFRWMNCLLMREISVKNTIRMWDTYLAEEQGFSEFHLYVCAALLVKWSDKLVKMDFQEVMMFLQSLPTKTWTEKDIELLLSEAFIWQSLYKGSAAHLKGQPQRPLLGNLQL

SEQ ID NO: 9 - DNA, 2959 bp, Trichoderma reesei
  CDS (1)..(44): CBS_FS8_ORF encodes putative mating polypeptide
atgacggaagctgagaacttcgaggacgatctcttcgccgatctgtaaggccttctgactccaggccctgcttccttccgatgtgctgacctgacgactagctacgaagatactgacgcaaagcctgccgccccggtcgcgagtgcggctcagaatgctgacacaaaggtccacgaccagcccgaaccagccgctgccccagctcccgctggcgacgaggacaactctggctacgaagccatgaaccaggatgcagatgatgatgaagatgaagtggacttcaacttgggaggcggcggcccttcaaacaataccaatgcgaacccgcaagacgacgcgccagcgactccgccctacgggaccgttcacaaagccagtgctaaggaagatgggtgagtttcccgtgttatgtgaagttatgatattggtgcatgagttcttttacattacctgcgacaagtgtcgtgcgcaatgttgccatatgatgatgagaggcttccagccagcgcgtaatcctgcaccttgcaaccacatgatggtatacacctgcttagagcaagtggctggcttgattatcggggagaagagtcagattcagcggcgggacaaggcccctggtcgaccggagagcaaaaatatatcaacaagcatgagagcgcggttggaaatatccagaaacctcctggaggagaatacagtcgaggcgccatcccggctgattaagtttcccggggacaatggagcaaagggagacgatggcgcttcgtccatcgacgaggctggggaaatccaagctttgactaagctgagaaagatatgcacccgcccgacgacctatttttcactccagtatgaacacgatgcttgtccttcatagctctcggtcgcagaggtctcaaagatagccagacgagagcatatgcgcttctcggaggctgtagcaagattgacagcgggtagcgcttagggtcaactcgtacctagcaacagacagtcagagacagagtcaagagcgatagcacacagacgaaagatcagaaaagagaaaggaaacaaatacgacacttgtctggcaccttgcgtccctctgcaatcgctgtatgcccttggctgtctcaccaagcatcgcatccgcgggggcctatgaagcgtcatcgttgacaactctttgacataacctcaactttatcctgtcgcaaagtaaagaggcaatgcatcggatccttgccgcatcaaacctcgaagtctcggcaacccgtcacgcctcttgaggctatccgcatacacttttcgagaattggtccgcatacgacccggcaacaggggcaaagcgcatggctttggcggatatttgcgtgcatcctcagctggccggccttgcccaacggccccgacgtgttgacagcgacgacgtatctggctccatggacttgacaatggcccctgtccatacgccgaagcaggccggcagttgctgcatctctcatatcatatgtcctgccccgagatctctcttctgttgccccatacgctactactgctaggatctcgtttcgctctgttgctttcttatgtttgccatgttatttgcgaggagactcttttgcgatgaattgacgtgctgacctttcgtttacttaggaaaatgttcatcggtggactgaactgggagacaacagaccgtacgtcttactacacactcttggctgaccttgcagatgctaacggacggtagaatccctgcgagattacttttcacaattcggagaagtcgtcgagtgcactgtgatgagagacagcagctccggccggtcccgtggctttggcttcttgacctttaaggaccccaagacggtcaatattgtcatggtcaaggaacattttctggatggcaagattgtaagctttttgttacactcaagcaagtgtccgatttactaacggacaatagattgatcccaaaagagctattccccgtgacgagcaggagaagacgagcaagatatttgtgggaggagtaagccaggacaccactgaccaggagttcagagagtactttgcccagtttggtcgagtagtcgacgccacattgatgatggacaaggacaccggcaggcctcgtggcttcggctttgtcacctttgagagcgaagcgggcgtggacgcctgtatcaatgtgccgctcgagatccatgggaagcctattgaggttaagaaggctcagccgagaggcaacctacgagaggaggaagaggcgtccaagcgaggaaagttccgaaagggcgatgaccagtctgtccaaggcggtatgggtcagcagatgggcggcaacaactcggccatgacgccgcagctcatggctcaatacttccagcgtatgcagcagtactttgccatgatgcagtcgcagatggccatgtccagaggcatgccaatgaacccggccatgtggcaacagatgcaacagatgcagcagatgcagcagatgatgggccgcggcggcggtcctggacagaacatgatgggccagatgaacccccagatgatgcagcagatgcaacagatgcagcagcagatgatgggccaacagggaggccagggcaacggtgacggcggctacaacggctacgaccagtaccaacagcaggcctctcagggtccccacggcggccgacgcggtggccgcggaaattatggaggtggcggatacgggatgggaggaggaggcggaggagcccccacgtcctgggagggcatgtacgacgatgtgccgcagccgaatgcgaaccaggggtcgtctcggcgcggcggaagcgtcggccacggaggaggctcggatgcccagcactctccgccagcaaacgccccgaccgggcccaagaatgccgggaagcctggcgccaactaccgcggcggaggacgaggcggaaacaggggcttccatccttatgcccgatga

SEQ ID NO: 10 - protein, 512 aa, Trichoderma reesei
MTEAENFEDDLFADLEDTDAKPAAPVASAAQNADTKVHDQPEPAAAPAPAGDEDNSGYEAMNQDADDDEDEVDFNLGGGGPSNNTNANPQDDAPATPPYGTVHKASAKEDGKMFIGGLNWETKSLRDYFSQFGEVVECTVMRDSSSGRSRGFGFLTFKDPKTVNIVMVKEHFLDGKIIDPKRAIPRDEQEKTSKIFVGGVSQDTTDQEFREYFAQFGRVVDATLMMDKDTGRPRGFGFVTFESEAGVDACINVPLEIHGKPIEVKKAQPRGNLREEEEASKRGKFRKGDDQSVQGGMGQQMGGNNSAMTPQLMAQYFQRMQQYFAMMQSQMAMSRGMPMNPAMWQQMQQMQQMQQMMGRGGGPGQNMMGQMNPQMMQQMQQMQQQMMGQQGGQGNGDGGYNGYDQYQQQASQGPHGGRRGGRGNYGGGGYGMGGGGGGAPTSWEGMYDDVPQPNANQGSSRRGGSVGHGGGSDAQHSPPANAPTGPKNAGKPGANYRGGGRGGNRGFHPYAR

SEQ ID NO: 11 - DNA, 3609 bp, Trichoderma reesei
  CDS (1)..(444): CBS_FS9_ORF encodes putative mating polypeptide
atggggagcctcaggaacattatgaatgtcgatgacgaccacgttgactcgcacgccataagaagggaccggaagccggcttcgagaacctcgctcgatcacgatctatccgtccccatggccagatacaatcatcacatcccggtggatctgtcaaggcaaacatcacctcctcacatcctccaccctgcagcgccaccactaggatataacaaccaccctggcgctcgccgacgcagcaacactagcaccgactccatggactcttcctacggacagagccagagccatacagcctactaccaagcaacagcaatgaggcccatcatgccaggaaccgtttcaggggatcaccctgtcaagctgacgcccatcactgggagggtcagtcgcgcgaagaagggagttcctgttcatacctgtgatgcctgccggccgccaaaggtatttcgcacattcactgcttcactcgttcggttccgatgttgttttgttgtaccatatgaagctaacattcgggggctcttctttcctcacattcaagacgttcacaagagcagagcatctacggtatgtcgtctacctcatcactccctacattcacaaacagcgttgctgacggtattacccctcctcagacgacatcaacttagccatcagccaccggagctctcctgcacggtctcgggctgcaagaaggtcttttatcgcaaagacctactggaccgacacttacagagacagtaaggcttctcatccattctccccatctcatctcagcatggaggggcggcagctgaacatgagatgatgtagtggcgaacacgacggcaaactgatcagagactcatcacgacggcgacgctccgacagcagtccatctcgaccatacagcagctcaccgccagcgcccgcgatgcaacaagcttcctctccatacagcgttggcgctgccgccacgcctgcttccagtgtcgggatggtggcgggccactggtcgtccgtcaacaggtcaacgccgccagggcctacaatgcaatcaccacacatccgagatgcgcagagtgcctatcacatggcagatatgagcgtggtcgaccccgtcacggccggcgtggtctctacctacagtgaaccgcggcctgccggactgggtgtaatcgacccccaagttacagagctgtgtatacctgaagcgactccgtcgaacattacatggacgacggacagctcgggcatcccgtcgacctcttcaggaagtgcttactcgacgccggcatccgtcagctccagttttcaacagtcgtcgacgcgagcccctgccgcggagtggactggaccgatgccctcttacaccacatccccgagccccgtcatcacggacaatggatatcccatgtcctttggctacgctgccacccctccacaggtctattcgtcagtctacggggatggcatgggaaccccctttgctggctttgaacacggcgcaaatctctacagcccccaaatgcttgacacgacagtccgcagcatgtcccctccagagctgatggtgggccagtccgccgagactctggtcgcggcgccggctgcgttgcctgtggatcgcatgatgtatcctcgcacctgcagccgcgagcctgtcgatgctcttggcctgtacacccttatgccgacagctctcagccatggcattcgaagcatgatcccgacgtacattgaggtgtactgggacaaggtccattccatgtaccccatcattcataaaccgacattcgagaacccgacaaacatgcccgaggagcacctggagattctccagtgcgccatggcggccattgcaacccaattcctcgagcatgaggagcaccgcgccaacgggcaccagctacatacctatgcgatggacaaggcgaagctggtaagtgccgaacaagaacaacaagattcggctgatgtcggaagagcagccagctaacctacccttctctcaaatagtatgccaactctggcaccccggaatggccattgcccattatgcaggctacgcttttgtgcgagtactatgctcgcttccggggacggaacaagaaggcatatatgccgtcgcctcgctttgaggcactgtaccaaatggttagttcccgctagatttctcatggttctctacgttcacatgccgcttggcatctctgagtgaacgggtttctctttctctttttctctttctctttttcttctctttgggcctttcctgcttcacgattttcctgtcttgtgtacaatcatgtccgcaaaaacaaaaacaaaagttcctacagcagtctcttttttctttcttttgctttcactttatgattattatgcttcccttctcccccttttttttatcttgacttgtttccaactggtgcttcttccttcttcttctactttcatcgtactctgccggggagacaacccaaccatctttattatctcttgctcattgtaccttcaatgcaggtcattcacgcacaaaccacttacccatcgactgtgggaccgtgcgatgcctctcagcaatggaggatctgggtccacatggaatcaaagaggcgcctcctatccgcatgcttcttgcttgatgtccatgcaatgtccttcctggagcaacctcgagccgcggttctcggcctggactatagtgatcctcggacactcccgatccccttgtcgatggccacgctgcagctgtgggatgcgcaaaactaccaggaatggagtagcatcagccagccagccatgccagcgactgttggggcgacgattctcgaaacaatcaccgcagcggatattgcttcgatcccggcctttgacgcttcgctgtttttggtcgcatacgttttacagctgccccagcgacggtccctcaccaagatcaacctcctagatgatgcatcgagcatcagcatgagccactttagaatcatcaatctgttcccgtactcgcccattgccatgtcgcatctcgccctccactacaccccgctgcacacgctcctgtcagtgtcgggcgacagctgggtcttcaacaagaaggtgctccaggccaccgacttcatggagcaccagaggcagctggagaagtggcgagattctggcagcgccgccgttgcggttgccttcgcctcacgggcgctcaagctgttcctaggactgcagaggtgcgtgagcaaggacgggagcaccgccgcctcgatccccacccccttccaacagcgcgcgcagcagcagcagcgcaaggagatttccgacttttggggcatctacgtgtgctccctcatctgctgggcctttggccatgtcggcgccgccagccgctccgaggccagcaaggcgccgacgcggagggccgccgtgcagtggatcctcaaggcgtcggacatggagcccgggcgcatccagcagatgtcggagcaggagaggcaggccggctccggggcggtcaatctcgcaagggcgtcgctcgagaaggattgcctgggcgggcggagcatcttgctcgccgatgccgtgggcgttttgaagaagttggaggagggggacaattatagacggttctag

SEQ ID NO: 12 - protein, 911 aa, Trichoderma reesei
MGSLRNIMNVDDDHVDSHAIRRDRKPASRTSLDHDLSVPMARYNHHIPVDLSRQTSPPHILHPAAPPLGYNNHPGARRRSNTSTDSMDSSYGQSQSHTAYYQATAMRPIMPGTVSGDHPVKLTPITGRVSRAKKGVPVHTCDACRPPKPPELSCTVSGCKKVFYRKDLLDRHLQRHGEHDGKLIRDSSRRRRSDSSPSRPYSSSPPAPAMQQASSPYSVGAAATPASSVGMVAGHWSSVNRSTPPGPTMQSPHIRDAQSAYHMADMSVVDPVTAGVVSTYSEPRPAGLGVIDPQVTELCIPEATPSNITWTTDSSGIPSTSSGSAYSTPASVSSSFQQSSTRAPAAEWTGPMPSYTTSPSPVITDNGYPMSFGYAATPPQVYSSVYGDGMGTPFAGFEHGANLYSPQMLDTTVRSMSPPELMVGQSAETLVAAPAALPVDRMMYPRTCSREPVDALGLYTLMPTALSHGIRSMIPTYIEVYWDKVHSMYPIIHKPTFENPTNMPEEHLEILQCAMAAIATQFLEHEEHRANGHQLHTYAMDKAKLVIHAQTTYPSTVGPCDASQQWRIWVHMESKRRLLSACFLLDVHAMSFLEQPRAAVLGLDYSDPRTLPIPLSMATLQLWDAQNYQEWSSISQPAMPATVGATILETITAADIASIPAFDASLFLVAYVLQLPQRRSLTKINLLDDASSISMSHFRIINLFPYSPIAMSHLALHYTPLHTLLSVSGDSWVFNKKVLQATDFMEHQRQLEKWRDSGSAAVAVAFASRALKLFLGLQRCVSKDGSTAASIPTPFQQRAQQQQRKEISDFWGIYVCSLICWAFGHVGAASRSEASKAPTRRAAVQWILKASDMEPGRIQQMSEQERQAGSGAVNLARASLEKDCLGGRSILLADAVGVLKKLEEGDNYRRF

SEQ ID NO: 13 - DNA, 20 bp, artificial sequence
  Primer
ggcacagctttcgtgatgaa

SEQ ID NO: 14 - DNA, 20 bp, artificial sequence
  Primer
tgctatacggcatccgaagg

SEQ ID NO: 15 - DNA, 20 bp, artificial sequence
  Primer
cgcaatcgcaatcgcaacaa

SEQ ID NO: 16 - DNA, 20 bp, artificial sequence
  Primer
tatagcgggcaatggtctca

SEQ ID NO: 17 - DNA, 20 bp, artificial sequence
  Primer
aactcgatgacgctgagcta

SEQ ID NO: 18 - DNA, 20 bp, artificial sequence
  Primer
agttgatgtaccaccccaga

SEQ ID NO: 19 - DNA, 20 bp, artificial sequence
  Primer
tcaacagcagcagacgaaca

SEQ ID NO: 20 - DNA, 20 bp, artificial sequence
  Primer
ctctgctgaagctgatgccg

SEQ ID NO: 21 - DNA, 20 bp, artificial sequence
  Primer
tgcaacagaacccccgagga

SEQ ID NO: 22 - DNA, 20 bp, artificial sequence
  Primer
ccttgaggaaagtcaggggc

SEQ ID NO: 23 - DNA, 20 bp, artificial sequence
  Primer
ttgctcacgacttgagcata

SEQ ID NO: 24 - DNA, 20 bp, artificial sequence
  Primer
ttcttggctcgcctgtgcgg

SEQ ID NO: 25 - DNA, 20 bp, artificial sequence
  Primer
accgcacttcaatcgcttgg

SEQ ID NO: 26 - DNA, 20 bp, artificial sequence
  Primer
ttcgttgagggggtggcgta

SEQ ID NO: 27 - DNA, 20 bp, artificial sequence
  Primer
ttcgtccatcgacgaggctg

SEQ ID NO: 28 - DNA, 20 bp, artificial sequence
  Primer
caaagagttgtcaacgatga

SEQ ID NO: 29 - DNA, 20 bp, artificial sequence
  Primer
gggcgctcaagctgttccta

SEQ ID NO: 30 - DNA, 20 bp, artificial sequence
  Primer
tcaaaacgcccacggcatcg

SEQ ID NO: 31 - DNA, 20 bp, artificial sequence
  Primer
cgatgtctcgggccatggaa

SEQ ID NO: 32 - DNA, 20 bp, artificial sequence
  Primer
agctccgaaatttcaagcaa

SEQ ID NO: 33 - DNA, 26 bp, artificial sequence
  Primer
cgctataccaagagctgtcattaatg

SEQ ID NO: 34 - DNA, 22 bp, artificial sequence
  Primer
tcgctgggcatgctgcagggaa

SEQ ID NO: 35 - DNA, 24 bp, artificial sequence
  Primer
gcacactctcgaatcaacagaaaa

SEQ ID NO: 36 - DNA, 20 bp, artificial sequence
  Primer
tggtaaaggatttgtacggg

SEQ ID NO: 37 - DNA, 20 bp, artificial sequence
  Primer
gttccgtcacgatgaagagg

SEQ ID NO: 38 - DNA, 20 bp, artificial sequence
  Primer
gctgggcagacggatcttaa

SEQ ID NO: 39 - DNA, 20 bp, artificial sequence
  Primer
acaggatgcactccaggtca

SEQ ID NO: 40 - DNA, 20 bp, artificial sequence
  Primer
tgagaccgtgcgagtcgatg

SEQ ID NO: 41 - DNA, 24 bp, artificial sequence
  Primer
ttcctgcggtggtgacaacctcca

SEQ ID NO: 42 - DNA, 24 bp, artificial sequence
  Primer
tagacgcggccaatcttctcgcga

SEQ ID NO: 43 - DNA, 20 bp, artificial sequence
  Primer
aagtcaccaaatacttctcg

SEQ ID NO: 44 - DNA, 18 bp, artificial sequence
  Primer
gtcggcatcgcactgcaa

SEQ ID NO: 45 - DNA, 20 bp, artificial sequence
  Primer
gattcggcgtctccattgcg

SEQ ID NO: 46 - DNA, 20 bp, artificial sequence
  Primer
tgttgtacatggctagggag

SEQ ID NO: 47 - DNA, 20 bp, artificial sequence
  Primer
ggcacagctttcgtgatgaa

SEQ ID NO: 48 - DNA, 20 bp, artificial sequence
  Primer
tgctatacggcatccgaagg

SEQ ID NO: 49 - DNA, 20 bp, artificial sequence
  Primer
cgcaatcgcaatcgcaacaa

SEQ ID NO: 50 - DNA, 20 bp, artificial sequence
  Primer
tatagcgggcaatggtctca

SEQ ID NO: 51 - DNA, 20 bp, artificial sequence
  Primer
aactcgatgacgctgagcta

SEQ ID NO: 52 - DNA, 20 bp, artificial sequence
  Primer
agttgatgtaccaccccaga

SEQ ID NO: 53 - DNA, 20 bp, artificial sequence
  Primer
tcaacagcagcagacgaaca

SEQ ID NO: 54 - DNA, 20 bp, artificial sequence
  Primer
ctctgctgaagctgatgccg

SEQ ID NO: 55 - DNA, 20 bp, artificial sequence
  Primer
tgcaacagaacccccgagga

SEQ ID NO: 56 - DNA, 20 bp, artificial sequence
  Primer
ccttgaggaaagtcaggggc

SEQ ID NO: 57 - DNA, 20 bp, artificial sequence
  Primer
ttgctcacgacttgagcata

SEQ ID NO: 58 - DNA, 20 bp, artificial sequence
  Primer
ttcttggctcgcctgtgcgg

SEQ ID NO: 59 - DNA, 20 bp, artificial sequence
  Primer
accgcacttcaatcgcttgg

SEQ ID NO: 60 - DNA, 20 bp, artificial sequence
  Primer
ttcgttgagggggtggcgta

SEQ ID NO: 61 - DNA, 20 bp, artificial sequence
  Primer
ttcgtccatcgacgaggctg

SEQ ID NO: 62 - DNA, 20 bp, artificial sequence
  Primer
caaagagttgtcaacgatga

SEQ ID NO: 63 - DNA, 20 bp, artificial sequence
  Primer
gggcgctcaagctgttccta

SEQ ID NO: 64 - DNA, 20 bp, artificial sequence
  Primer
tcaaaacgcccacggcatcg

SEQ ID NO: 65 - DNA, 20 bp, artificial sequence
  Primer
cgatgtctcgggccatggaa

SEQ ID NO: 66 - DNA, 20 bp, artificial sequence
  Primer
agctccgaaatttcaagcaa

SEQ ID NO: 67 - DNA, 26 bp, artificial sequence
  Primer
cgctataccaagagctgtcattaatg

SEQ ID NO: 68 - DNA, 22 bp, artificial sequence
  Primer
tcgctgggcatgctgcagggaa

SEQ ID NO: 69 - DNA, 24 bp, artificial sequence
  Primer
gcacactctcgaatcaacagaaaa

SEQ ID NO: 70 - DNA, 20 bp, artificial sequence
  Primer
tggtaaaggatttgtacggg

SEQ ID NO: 71 - DNA, 20 bp, artificial sequence
  Primer
gttccgtcacgatgaagagg

SEQ ID NO: 72 - DNA, 20 bp, artificial sequence
  Primer
gctgggcagacggatcttaa

SEQ ID NO: 73 - DNA, 20 bp, artificial sequence
  Primer
acaggatgcactccaggtca

SEQ ID NO: 74 - DNA, 20 bp, artificial sequence
  Primer
tgagaccgtgcgagtcgatg

SEQ ID NO: 75 - DNA, 24 bp, artificial sequence
  Primer
ttcctgcggtggtgacaacctcca

SEQ ID NO: 76 - DNA, 24 bp, artificial sequence
  Primer
tagacgcggccaatcttctcgcga

SEQ ID NO: 77 - DNA, 20 bp, artificial sequence
  Primer
aagtcaccaaatacttctcg

SEQ ID NO: 78 - DNA, 18 bp, artificial sequence
  Primer
gtcggcatcgcactgcaa

SEQ ID NO: 79 - DNA, 20 bp, artificial sequence
  Primer
gattcggcgtctccattgcg

SEQ ID NO: 80 - DNA, 20 bp, artificial sequence
  Primer
tgttgtacatggctagggag